SEQUENCE LISTING (135 sequences)

SEQ ID NO: 1 - DNA, 15 bp, Artificial Sequence
  Synthetic Sequence
gctagacgttagcgt

SEQ ID NO: 2 - DNA, 15 bp, Artificial Sequence
  Synthetic Sequence
gctagatgttagcgt

SEQ ID NO: 3 - DNA, 15 bp, Artificial Sequence
  Synthetic Sequence
gctagacgttagcgt

SEQ ID NO: 4 - DNA, 15 bp, Artificial Sequence
  Synthetic Sequence
gctagacgttagcgt

SEQ ID NO: 5 - DNA, 15 bp, Artificial Sequence
  Synthetic Sequence
gcatgacgttgagct

SEQ ID NO: 6 - DNA, 20 bp, Artificial Sequence
  Synthetic Sequence
atggaaggtccagcgttctc

SEQ ID NO: 7 - DNA, 20 bp, Artificial Sequence
  Synthetic Sequence
atcgactctcgagcgttctc

SEQ ID NO: 8 - DNA, 20 bp, Artificial Sequence
  Synthetic Sequence
atcgactctcgagcgttctc

SEQ ID NO: 9 - DNA, 20 bp, Artificial Sequence
  Synthetic Sequence
atcgactctcgagcgttctc

SEQ ID NO: 10 - DNA, 20 bp, Artificial Sequence
  Synthetic Sequence
atggaaggtccaacgttctc

SEQ ID NO: 11 - DNA, 20 bp, Artificial Sequence
  Synthetic Sequence
gagaacgctggaccttccat

SEQ ID NO: 12 - DNA, 20 bp, Artificial Sequence
  Synthetic Sequence
gagaacgctcgaccttccat

SEQ ID NO: 13 - DNA, 20 bp, Artificial Sequence
  Synthetic Sequence
gagaacgctcgaccttcgat

SEQ ID NO: 14 - DNA, 20 bp, Artificial Sequence
  Synthetic Sequence
gagaacgctggaccttccat

SEQ ID NO: 15 - DNA, 20 bp, Artificial Sequence
  Synthetic Sequence
gagaacgatggaccttccat

SEQ ID NO: 16 - DNA, 20 bp, Artificial Sequence
  Synthetic Sequence
gagaacgctccagcactgat

SEQ ID NO: 17 - DNA, 20 bp, Artificial Sequence
  Synthetic Sequence
tccatgtcggtcctgatgct

SEQ ID NO: 18 - DNA, 20 bp, Artificial Sequence
  Synthetic Sequence
tccatgtcggtcctgatgct

SEQ ID NO: 19 - DNA, 20 bp, Artificial Sequence
  Synthetic Sequence
tccatgacgttcctgatgct

SEQ ID NO: 20 - DNA, 20 bp, Artificial Sequence
  Synthetic Sequence
tccatgtcggtcctgctgat

SEQ ID NO: 21 - DNA, 8 bp, Artificial Sequence
  Synthetic Sequence
tcaacgtt

SEQ ID NO: 22 - DNA, 8 bp, Artificial Sequence
  Synthetic Sequence
tcagcgct

SEQ ID NO: 23 - DNA, 8 bp, Artificial Sequence
  Synthetic Sequence
tcatcgat

SEQ ID NO: 24 - DNA, 8 bp, Artificial Sequence
  Synthetic Sequence
tcttcgaa

SEQ ID NO: 25 - DNA, 7 bp, Artificial Sequence
  Synthetic Sequence
caacgtt

SEQ ID NO: 26 - DNA, 8 bp, Artificial Sequence
  Synthetic Sequence
ccaacgtt

SEQ ID NO: 27 - DNA, 8 bp, Artificial Sequence
  Synthetic Sequence
aacgttct

SEQ ID NO: 28 - DNA, 8 bp, Artificial Sequence
  Synthetic Sequence
tcaacgtc

SEQ ID NO: 29 - DNA, 20 bp, Artificial Sequence
  Synthetic Sequence
atggactctccagcgttctc

SEQ ID NO: 30 - DNA, 20 bp, Artificial Sequence
  Synthetic Sequence
atggaaggtccaacgttctc

SEQ ID NO: 31 - DNA, 20 bp, Artificial Sequence
  Synthetic Sequence
atcgactctcgagcgttctc

SEQ ID NO: 32 - DNA, 20 bp, Artificial Sequence
  Synthetic Sequence
atggaggctccatcgttctc

SEQ ID NO: 33 - DNA, 20 bp, Artificial Sequence
  Synthetic Sequence
atcgactctcgagcgttctc

SEQ ID NO: 34 - DNA, 20 bp, Artificial Sequence
  Synthetic Sequence
atcgactctcgagcgttctc

SEQ ID NO: 35 - DNA, 20 bp, Artificial Sequence
  Synthetic Sequence
tccatgtcggtcctgatgct

SEQ ID NO: 36 - DNA, 20 bp, Artificial Sequence
  Synthetic Sequence
tccatgccggtcctgatgct

SEQ ID NO: 37 - DNA, 20 bp, Artificial Sequence
  Synthetic Sequence
tccatggcggtcctgatgct

SEQ ID NO: 38 - DNA, 20 bp, Artificial Sequence
  Synthetic Sequence
tccatgacggtcctgatgct

SEQ ID NO: 39 - DNA, 20 bp, Artificial Sequence
  Synthetic Sequence
tccatgtcgatcctgatgct

SEQ ID NO: 40 - DNA, 20 bp, Artificial Sequence
  Synthetic Sequence
tccatgtcgctcctgatgct

SEQ ID NO: 41 - DNA, 20 bp, Artificial Sequence
  Synthetic Sequence
tccatgtcgtccctgatgct

SEQ ID NO: 42 - DNA, 20 bp, Artificial Sequence
  Synthetic Sequence
tccatgacgtgcctgatgct

SEQ ID NO: 43 - DNA, 20 bp, Artificial Sequence
  Synthetic Sequence
tccataacgttcctgatgct

SEQ ID NO: 44 - DNA, 20 bp, Artificial Sequence
  Synthetic Sequence
tccatgacgtccctgatgct

SEQ ID NO: 45 - DNA, 20 bp, Artificial Sequence
  Synthetic Sequence
tccatcacgtgcctgatgct

SEQ ID NO: 46 - DNA, 19 bp, Artificial Sequence
  Synthetic Sequence
ggggtcaacgttgacgggg

SEQ ID NO: 47 - DNA, 19 bp, Artificial Sequence
  Synthetic Sequence
ggggtcagtcgtgacgggg

SEQ ID NO: 48 - DNA, 15 bp, Artificial Sequence
  Synthetic Sequence
gctagacgttagtgt

SEQ ID NO: 49 - DNA, 20 bp, Artificial Sequence
  Synthetic Sequence
tccatgtcgttcctgatgct

SEQ ID NO: 50 - DNA, 24 bp, Artificial Sequence
  Synthetic Sequence
accatggacgatctgtttcccctc

SEQ ID NO: 51 - DNA, 18 bp, Artificial Sequence
  Synthetic Sequence
tctcccagcgtgcgccat

SEQ ID NO: 52 - DNA, 24 bp, Artificial Sequence
  Synthetic Sequence
accatggacgaactgtttcccctc

SEQ ID NO: 53 - DNA, 24 bp, Artificial Sequence
  Synthetic Sequence
accatggacgagctgtttcccctc

SEQ ID NO: 54 - DNA, 24 bp, Artificial Sequence
  Synthetic Sequence
accatggacgacctgtttcccctc

SEQ ID NO: 55 - DNA, 24 bp, Artificial Sequence
  Synthetic Sequence
accatggacgtactgtttcccctc

SEQ ID NO: 56 - DNA, 24 bp, Artificial Sequence
  Synthetic Sequence
accatggacggtctgtttcccctc

SEQ ID NO: 57 - DNA, 24 bp, Artificial Sequence
  Synthetic Sequence
accatggacgttctgtttcccctc

SEQ ID NO: 58 - DNA, 15 bp, Artificial Sequence
  Synthetic Sequence
cacgttgaggggcat

SEQ ID NO: 59 - DNA, 12 bp, Artificial Sequence
  Synthetic Sequence
tcagcgtgcgcc

SEQ ID NO: 60 - DNA, 17 bp, Artificial Sequence
  Synthetic Sequence
atgacgttcctgacgtt

SEQ ID NO: 61 - DNA, 17 bp, Artificial Sequence
  Synthetic Sequence
tctcccagcgggcgcat

SEQ ID NO: 62 - DNA, 20 bp, Artificial Sequence
  Synthetic Sequence
tccatgtcgttcctgtcgtt

SEQ ID NO: 63 - DNA, 20 bp, Artificial Sequence
  Synthetic Sequence
tccatagcgttcctagcgtt

SEQ ID NO: 64 - DNA, 21 bp, Artificial Sequence
  Synthetic Sequence
tcgtcgctgtctccccttctt

SEQ ID NO: 65 - DNA, 19 bp, Artificial Sequence
  Synthetic Sequence
tcctgacgttcctgacgtt

SEQ ID NO: 66 - DNA, 19 bp, Artificial Sequence
  Synthetic Sequence
tcctgtcgttcctgtcgtt

SEQ ID NO: 67 - DNA, 20 bp, Artificial Sequence
  Synthetic Sequence
tccatgtcgtttttgtcgtt

SEQ ID NO: 68 - DNA, 20 bp, Artificial Sequence
  Synthetic Sequence
tcctgtcgttccttgtcgtt

SEQ ID NO: 69 - DNA, 20 bp, Artificial Sequence
  Synthetic Sequence
tccttgtcgttcctgtcgtt

SEQ ID NO: 70 - DNA, 20 bp, Artificial Sequence
  Synthetic Sequence
tcctgtcgttttttgtcgtt

SEQ ID NO: 71 - DNA, 21 bp, Artificial Sequence
  Synthetic Sequence
tcgtcgctgtctgcccttctt

SEQ ID NO: 72 - DNA, 21 bp, Artificial Sequence
  Synthetic Sequence
tcgtcgctgttgtcgtttctt

SEQ ID NO: 73 - DNA, 20 bp, Artificial Sequence
  Synthetic Sequence
tccatgcgtgcgtgcgtttt

SEQ ID NO: 74 - DNA, 20 bp, Artificial Sequence
  Synthetic Sequence
tccatgcgttgcgttgcgtt

SEQ ID NO: 75 - DNA, 20 bp, Artificial Sequence
  Synthetic Sequence
tccacgacgttttcgacgtt

SEQ ID NO: 76 - DNA, 20 bp, Artificial Sequence
  Synthetic Sequence
tcgtcgttgtcgttgtcgtt

SEQ ID NO: 77 - DNA, 24 bp, Artificial Sequence
  Synthetic Sequence
tcgtcgttttgtcgttttgtcgtt

SEQ ID NO: 78 - DNA, 22 bp, Artificial Sequence
  Synthetic Sequence
tcgtcgttgtcgttttgtcgtt

SEQ ID NO: 79 - DNA, 21 bp, Artificial Sequence
  Synthetic Sequence
gcgtgcgttgtcgttgtcgtt

SEQ ID NO: 80 - DNA, 21 bp, Artificial Sequence
  Synthetic Sequence
tgtcgtttgtcgtttgtcgtt

SEQ ID NO: 81 - DNA, 25 bp, Artificial Sequence
  Synthetic Sequence
tgtcgttgtcgttgtcgttgtcgtt

SEQ ID NO: 82 - DNA, 19 bp, Artificial Sequence
  Synthetic Sequence
tgtcgttgtcgttgtcgtt

SEQ ID NO: 83 - DNA, 14 bp, Artificial Sequence
  Synthetic Sequence
tcgtcgtcgtcgtt

SEQ ID NO: 84 - DNA, 13 bp, Artificial Sequence
  Synthetic Sequence
tgtcgttgtcgtt

SEQ ID NO: 85 - DNA, 20 bp, Artificial Sequence
  Synthetic Sequence
tccatagcgttcctagcgtt

SEQ ID NO: 86 - DNA, 20 bp, Artificial Sequence
  Synthetic Sequence
tccatgacgttcctgacgtt

SEQ ID NO: 87 - DNA, 6 bp, Artificial Sequence
  Synthetic Sequence
gtcgyt

SEQ ID NO: 88 - DNA, 7 bp, Artificial Sequence
  Synthetic Sequence
tgtcgyt

SEQ ID NO: 89 - DNA, 18 bp, Artificial Sequence
  Synthetic Sequence
agctatgacgttccaagg

SEQ ID NO: 90 - DNA, 20 bp, Artificial Sequence
  Synthetic Sequence
tccatgacgttcctgacgtt

SEQ ID NO: 91 - DNA, 20 bp, Artificial Sequence
  Synthetic Sequence
atcgactctcgaacgttctc

SEQ ID NO: 92 - DNA, 20 bp, Artificial Sequence
  Synthetic Sequence
tccatgtcggtcctgacgca

SEQ ID NO: 93 - DNA, 8 bp, Artificial Sequence
  Synthetic Sequence
tcttcgat

SEQ ID NO: 94 - DNA, 20 bp, Artificial Sequence
  Synthetic Sequence
ataggaggtccaacgttctc

SEQ ID NO: 95 - DNA, 15 bp, Artificial Sequence
  Synthetic Sequence
gctagaggggagggt

SEQ ID NO: 96 - DNA, 15 bp, Artificial Sequence
  Synthetic Sequence
gctagatgttagggg

SEQ ID NO: 97 - DNA, 15 bp, Artificial Sequence
  Synthetic Sequence
gctagaggggagggt

SEQ ID NO: 98 - DNA, 15 bp, Artificial Sequence
  Synthetic Sequence
gctagaggggagggt

SEQ ID NO: 99 - DNA, 15 bp, Artificial Sequence
  Synthetic Sequence
gcatgagggggagct

SEQ ID NO: 100 - DNA, 20 bp, Artificial Sequence
  Synthetic Sequence
atggaaggtccagggggctc

SEQ ID NO: 101 - DNA, 20 bp, Artificial Sequence
  Synthetic Sequence
atggactctggagggggctc

SEQ ID NO: 102 - DNA, 20 bp, Artificial Sequence
  Synthetic Sequence
atggactctggagggggctc

SEQ ID NO: 103 - DNA, 20 bp, Artificial Sequence
  Synthetic Sequence
atggactctggagggggctc

SEQ ID NO: 104 - DNA, 20 bp, Artificial Sequence
  Synthetic Sequence
atggaaggtccaaggggctc

SEQ ID NO: 105 - DNA, 20 bp, Artificial Sequence
  Synthetic Sequence
gagaaggggggaccttccat

SEQ ID NO: 106 - DNA, 20 bp, Artificial Sequence
  Synthetic Sequence
gagaaggggggaccttccat

SEQ ID NO: 107 - DNA, 20 bp, Artificial Sequence
  Synthetic Sequence
gagaaggggggaccttggat

SEQ ID NO: 108 - DNA, 20 bp, Artificial Sequence
  Synthetic Sequence
gagaaggggggaccttccat

SEQ ID NO: 109 - DNA, 20 bp, Artificial Sequence
  Synthetic Sequence
gagaaggggggaccttccat

SEQ ID NO: 110 - DNA, 20 bp, Artificial Sequence
  Synthetic Sequence
gagaaggggccagcactgat

SEQ ID NO: 111 - DNA, 20 bp, Artificial Sequence
  Synthetic Sequence
tccatgtggggcctgatgct

SEQ ID NO: 112 - DNA, 20 bp, Artificial Sequence
  Synthetic Sequence
tccatgtggggcctgatgct

SEQ ID NO: 113 - DNA, 20 bp, Artificial Sequence
  Synthetic Sequence
tccatgaggggcctgatgct

SEQ ID NO: 114 - DNA, 20 bp, Artificial Sequence
  Synthetic Sequence
tccatgtggggcctgctgat

SEQ ID NO: 115 - DNA, 20 bp, Artificial Sequence
  Synthetic Sequence
atggactctccggggttctc

SEQ ID NO: 116 - DNA, 20 bp, Artificial Sequence
  Synthetic Sequence
atggaaggtccggggttctc

SEQ ID NO: 117 - DNA, 20 bp, Artificial Sequence
  Synthetic Sequence
atggactctggaggggtctc

SEQ ID NO: 118 - DNA, 20 bp, Artificial Sequence
  Synthetic Sequence
atggaggctccatggggctc

SEQ ID NO: 119 - DNA, 20 bp, Artificial Sequence
  Synthetic Sequence
atggactctggggggttctc

SEQ ID NO: 120 - DNA, 20 bp, Artificial Sequence
  Synthetic Sequence
atggactctggggggttctc

SEQ ID NO: 121 - DNA, 20 bp, Artificial Sequence
  Synthetic Sequence
tccatgtgggtggggatgct

SEQ ID NO: 122 - DNA, 20 bp, Artificial Sequence
  Synthetic Sequence
tccatgcgggtggggatgct

SEQ ID NO: 123 - DNA, 20 bp, Artificial Sequence
  Synthetic Sequence
tccatgggggtcctgatgct

SEQ ID NO: 124 - DNA, 20 bp, Artificial Sequence
  Synthetic Sequence
tccatgggggtcctgatgct

SEQ ID NO: 125 - DNA, 20 bp, Artificial Sequence
  Synthetic Sequence
tccatgtggggcctgatgct

SEQ ID NO: 126 - DNA, 20 bp, Artificial Sequence
  Synthetic Sequence
tccatgtggggcctgatgct

SEQ ID NO: 127 - DNA, 20 bp, Artificial Sequence
  Synthetic Sequence
tccatggggtccctgatgct

SEQ ID NO: 128 - DNA, 20 bp, Artificial Sequence
  Synthetic Sequence
tccatggggtgcctgatgct

SEQ ID NO: 129 - DNA, 20 bp, Artificial Sequence
  Synthetic Sequence
tccatggggttcctgatgct

SEQ ID NO: 130 - DNA, 20 bp, Artificial Sequence
  Synthetic Sequence
tccatggggtccctgatgct

SEQ ID NO: 131 - DNA, 20 bp, Artificial Sequence
  Synthetic Sequence
tccatcgggggcctgatgct

SEQ ID NO: 132 - DNA, 14 bp, Artificial Sequence
  Synthetic Sequence
gctagagggagtgt

SEQ ID NO: 133 - DNA, 20 bp, Artificial Sequence
  Synthetic Sequence
gggggggggggggggggggg

SEQ ID NO: 134 - DNA, 11 bp, Artificial Sequence
  Synthetic Sequence
gggngggnggg

SEQ ID NO: 135 - DNA, 10 bp, Artificial Sequence
  Synthetic Sequence
tcntnncgnn

(19) United States
(12) Patent Application Publication
(10) Pub. No.: US 2004/0131628 A1
(43) Pub. Date: Jul. 8, 2004

(54) Nucleic acids for the treatment of disorders associated with microorganisms

(76) Inventors: Robert L. Bratzler, Concord, MA (US); Deanna M. Petersen, Newton, MA (US)

Correspondence Address: Patrick R.H. Waller, Ph.D., 600 Atlantic Avenue, Boston, MA, 02210, US

(21) Appl. No.: 10/666,733

(22) Filed: Sep. 19, 2003

Related U.S. Application Data
(63) Continuation of application No. 09/801,839, filed on Mar. 8, 2001, now abandoned.
(60) Provisional application No. 60/187,834, filed on Mar. 8, 2000.

(51) Int. Cl. A61K 39/00; A61K 39/38

(52) U.S. Cl. 424/184.1

(57) ABSTRACT

The invention involves administration of an immunostimulatory nucleic acid alone or in combination with an anti-microbial agent for the treatment or prevention of infectious disease associated with microorganisms in subjects, for preventing antibiotic resistance and for treating and preventing warts. The combination of drugs are administered in synergistic amounts or in various dosages or at various time schedules. The invention also relates to kits and compositions concerning the combination of drugs.

### RELATED APPLICATIONS

This application claims priority to and is a continuation of co-pending U.S. Ser. No. 09/801,839 filed On Mar. 8, 2001, which claims priority under Title 35 §119(e) of the U.S. Provisional Application No. 60/187,834, filed Mar. 8, 2000, and entitled “Nucleic Acids for the Treatment of Disorders Associated with Microorganisms”, the entire contents of which are incorporated herein by reference.

### FIELD OF THE INVENTION

The present invention relates to the use of immunostimulatory nucleic acids in the treatment of microbial disorders (e.g., bacterial infections, viral infections, fungal infections, parasitic infections, etc.).

### BACKGROUND OF THE INVENTION

Infectious disease is one of the leading causes of death throughout the world. In the United States alone the death rate due to infectious disease rose 58% between 1980 and 1992. During this time, the use of anti-infective therapies to combat infectious disease has grown significantly and is now a multi-billion dollar a year industry. Even with these increases in anti-infective agent use, the treatment and prevention of infectious disease remains a challenge to the medical community throughout the world. In general, there are three types of anti-infective agents, anti-bacterial agents, anti-viral agents, and anti-fungal agents, and even within these classes of agents there is some overlap with respect to the type of microorganism they are useful for treating.

Anti-bacterial agents kill or inhibit bacteria, and include antibiotics as well as other synthetic or natural compounds having similar functions. Antibiotics are low molecular weight molecules which are produced as secondary metabolites by cells, such as microorganisms. In general, antibiotics interfere with one or more bacterial functions or structures which are specific for the microorganism and which are not present in host cells. Anti-viral agents, which can be isolated from natural sources or synthesized, are useful for killing or inhibiting viruses. Anti-fungal agents are used to treat superficial fungal infections as well as opportunistic and primary systemic fungal infections.

One of the problems with anti-infective therapies is the side effects occurring in the host that is treated with the anti-infective. For instance, many anti-infectious agents can kill or inhibit a broad spectrum of microorganisms and are not specific for a particular type of species. Treatment with these types of anti-infectious agents results in the killing of the normal microbial flora living in the host, as well as the infectious microorganism. The loss of the microbial flora can lead to disease complications and predispose the host to infection by other pathogens, since the microbial flora compete with and function as barriers to infectious pathogens. Other side effects may arise as a result of specific or non-specific effects of these chemical entities on non-microbial cells or tissues of the host.

Another problem with wide-spread use of anti-infectants is the development of antibiotic resistant strains of microorganisms. Already, vancomycin-resistant enterococci, penicillin-resistant pneumococci, multi-resistant S. aureus, and multi-resistant tuberculosis strains have developed and are becoming major clinical problems. Widespread use of anti-infectants will likely produce many antibiotic-resistant strains of bacteria. As a result, new anti-infective strategies will be required to combat these microorganisms.

### SUMMARY OF THE INVENTION

Improved methods and products for the prevention and/or treatment of infections associated with microorganisms are provided according to the invention. The invention is based, in some aspects, on the finding that when some immunostimulatory nucleic acids are used in conjunction with medicaments for the treatment of infectious disease, unexpected and improved results are observed. For instance, the efficacy of the combination of some immunostimulatory nucleic acids and anti-infectious disease medicaments is profoundly improved over the use of each of the medicaments alone. The results are surprising in part because the drugs act through different mechanisms and would not necessarily be expected to improve the efficacy of one another in a synergistic manner.

In one aspect, the invention provides a method for treating or preventing an infectious disease in a subject having or at risk of developing the infectious disease, comprising administering to a subject in need of such treatment a poly-G nucleic acid and an anti-microbial agent in an effective amount for treating or preventing the infectious disease. In an important embodiment, the poly-G nucleic acid is not conjugated to the anti-microbial agent. In one embodiment, the effective amount is a synergistic amount.

In this and other aspects of the invention, the poly-G nucleic acid may comprise the following formula: 5′ X₁X₂GGGX₃X₄ 3′, wherein X₁, X₂, X₃, and X₄ are nucleotides. In one embodiment, at least one of X₃ and X₄ are a G. In another embodiment, both of X₃ and X₄ are a G. The poly-G nucleic acid may additionally or alternatively comprise the following formula: 5′ GGGNGGG 3′, wherein N represents between 0 and 20 nucleotides. The poly-G nucleic acid may also be a nucleic acid that comprises the following formula: 5′ GGGNGGGNGGG 3′ (SEQ ID NO: 134), wherein N represents between 0 and 20 nucleotides.

In certain embodiments, the poly-G nucleic acid is administered mucosally, and in such embodiments, the poly-G nucleic acid preferably is free of unmethylated CG dinucleotides. Poly-G nucleic acids that are free of unmethylated CG dinucleotides may be selected from the group of nucleic acids having nucleotide sequences of SEQ ID NOs: 95-133. In other embodiments, the poly-G nucleic acid is administered systemically, and in such embodiment, the poly-G nucleic acid may comprise at least one unmethylated CG dinucleotide. Poly-G nucleic acids that comprise at least one unmethylated CG dinucleotide may be selected from the group of nucleic acids having a nucleotide sequences of SEQ ID NO 46, 47, 58, and 61.

In other embodiments, the poly-G nucleic acid has a phosphorothioate modified backbone, and the poly-G nucleic acid is administered systemically. In a related embodiment, the poly-G nucleic acid is free of T-rich motifs and methylated CpG motifs. As used herein, a methylated CpG motif is a CG dinucleotide in which the C residue is methylated.

In the several aspects of the invention unless otherwise stated, the anti-microbial agent is selected from the group consisting of an anti-bacterial agent, an anti-viral agent, an anti-fungal agent, and an anti-parasitic agent. In some embodiments, the anti-microbial agent is an anti-bacterial agent. In other embodiments, the anti-microbial agent is an anti-viral agent. In still other embodiments, the anti-microbial agent is an anti-fungal agent. Examples of each category of anti-microbial agent are provided herein. In some particular embodiments relating to the use of poly-G nucleic acids and CpG nucleic acids in the treatment and prevention of infectious disease, the anti-microbial agent is not a cytokine.

In certain embodiments, the anti-viral agent is selected from the group consisting of immunoglobulin, amantadine, interferon, nucleoside analogues, and protease inhibitors.

In other embodiments, the anti-bacterial agent is an antibiotic. In one embodiment, the anti-bacterial agent is a broad spectrum antibiotic. In another embodiment, the anti-bacterial agent is a narrow spectrum antibiotic. In yet a further embodiment, the anti-bacterial agent is a limited spectrum antibiotic. The anti-bacterial agent may be selected from the group consisting of cell wall synthesis inhibitors, cell membrane inhibitors, protein synthesis inhibitors, nucleic acid synthesis or functional inhibitors, and competitive inhibitors.

In some embodiments of the several aspects of the invention, the method further comprise administering an antigen, preferably a microbial antigen, to the subject. The microbial antigen may be selected from the group consisting of a bacterial antigen, a viral antigen, a fungal antigen, and a parasitic antigen. In some embodiments, the antigen is not conjugated to the immunostimulatory nucleic acid. In some particular embodiments, the antigen is not conjugated to a CpG nucleic acid, and in other embodiments, it is not conjugated to a poly-G nucleic acid.

In another aspect, the invention provides a method for treating or preventing an infectious disease in a subject having or at risk of developing the infectious disease, comprising administering to a subject in need of such treatment a CpG nucleic acid and an anti-microbial agent in an effective amount for treating or preventing the infectious disease, wherein the CpG nucleic acid is administered systemically. In important embodiment, the effective amount is a synergistic amount. The anti-microbial agent may be administered systemically or locally. In some embodiments in which an antigen is further administered to the subject, the antigen may be administered systemically or locally. In an important embodiment, the CpG nucleic acid is not a T-rich nucleic acid, not a methylated CpG nucleic acid, and not a Th2 immunostimulatory nucleic acid. As used herein, a Th2 immunostimulatory nucleic acid is a non-CpG nucleic acid (i.e., a nucleic acid lacking both a methylated and an unmethylated CG dinucleotide) that stimulates a Th2 immune response. In one embodiment, the CpG nucleic acid has a modified backbone such as a phosphorothioate modified backbone. In yet another embodiment, an adjuvant may be further administered to the subject, provided that the anti-microbial agent is selected from the group consisting of an anti-bacterial agent and an anti-fungal agent.

In yet another method for prophylactically treating a subject at risk of developing the infectious disease. The method comprises administering to a subject in need of such treatment an immunostimulatory nucleic acid having a phosphorothioate modified backbone, and an anti-microbial agent in an amount effective to inhibit the infectious disease. The immunostimulatory nucleic acid is free of a T-rich motif, an unmethylated CpG motif, and a methylated CpG motif. In important embodiments, the effective amount is a synergistic amount. In one embodiment, the immunostimulatory nucleic acid is administered systemically.

In yet another aspect, the invention provides a method for treating or preventing warts in a subject having or at risk of developing warts by administering to a subject in need of such treatment, an immunostimulatory nucleic acid that does not have a phosphorothioate modified backbone in an effective amount for treating or preventing the wart. In one embodiment, the immunostimulatory nucleic acid is a CpG nucleic acid. In another embodiment, the immunostimulatory nucleic acid is a poly-G nucleic acid. In still other embodiments, the immunostimulatory nucleic acid is a T-rich nucleic acid or a Th2 immunostimulatory nucleic acid. In certain embodiments, an anti-microbial agent, preferably an anti-viral agent, is administered to the subject. In these latter embodiments, the immunostimulatory nucleic acid and the anti-microbial agent can be administered in an effective amount to synergistically treat or prevent the wart.

In yet a further aspect, the invention provides a method for preventing antibiotic resistance by administering to a subject prior to, at the same time as or after the subject has received antibiotic therapy an effective amount of an immunostimulatory nucleic acid for preventing antibiotic resistance. In one embodiment, the immunostimulatory nucleic acid is a CpG nucleic acid. In another embodiment, the immunostimulatory nucleic acid is a T-rich nucleic acid. In still another embodiment, the immunostimulatory nucleic acid is a poly-G nucleic acid. The immunostimulatory nucleic acid may be a nucleic acid having a phosphorothioate backbone modification. In one embodiment, the immunostimulatory nucleic acid is administered before the antibiotic. In another embodiment, the immunostimulatory nucleic acid is administered at the same time as the antibiotic. In still another embodiment, the immunostimulatory nucleic acid is administered after the antibiotic. In important embodiments, the immunostimulatory nucleic acid is administered systemically.

In a further aspect, the invention provides a method for preventing an allergic reaction in a subject receiving an anti-microbial agent. The method comprises administering to a subject receiving an anti-microbial agent an immunostimulatory nucleic acid in an effective amount to prevent an allergic reaction to the anti-microbial agent. In an important embodiment, the anti-microbial agent is an anti-bacterial agent (e.g., penicillin). The immunostimulatory nucleic acid may be a CpG nucleic acid, a T-rich nucleic acid, or a poly-G nucleic acid. In one embodiment, the nucleic acid has a modified backbone (e.g., a phosphorothioate modified backbone).

In yet another aspect, the invention provides kits and compositions intended for use in the several afore-mentioned methods of the invention. One such kit comprises at least one container housing an immunostimulatory nucleic acid, and at least one container housing an anti-microbial agent, and instructions for systemic administration of the immunostimulatory nucleic acid. In this latter kit, the immunostimulatory nucleic acid is selected from the group consisting of a CpG nucleic acid, a poly-G nucleic acid and a nucleic acid having a phosphorothioate modified backbone. In still other kits which include only poly-G nucleic acids as the immunostimulatory nucleic acid, the instructions provided are for systemic or local delivery of the immunostimulatory nucleic acid. In important embodiments, the at least one container housing an immunostimulatory nucleic acid is a sustained release vehicle. The kits may further comprise instructions for administering the immunostimulatory nucleic acid and the anti-microbial agent in an effective amount for inducing a synergistic immune response in the subject.

In a further aspect, the invention provides a composition comprising an immunostimulatory nucleic acid and an antibiotic, formulated in a pharmaceutically-acceptable carrier and in an effective amount for preventing the development of antibiotic resistant strains of bacteria. In one embodiment, the antibiotic is selected from the group consisting of broad spectrum antibiotics, narrow spectrum antibiotics, and limited spectrum antibiotics.

Each of the limitations of the invention can encompass various embodiments of the invention. It is, therefore, anticipated that each of the limitations of the invention involving any one element or combinations of elements can be included in each aspect of the invention.

### DETAILED DESCRIPTION OF THE INVENTION

The invention relates to methods and products for the treatment of infectious disease using a combination of anti-microbial agents and some immunostimulatory nucleic acids. In some instances, the combination of anti-microbial agents and immunostimulatory nucleic acids is synergistic, resulting in greater than additive effects than would otherwise be expected using the agents separately. In other instances, the combination overcomes obstacles previously observed with particular anti-microbial agents, including microbial resistance and allergy to particular anti-microbial agents.

Depending upon the specific aspect of the invention being practiced, the anti-microbial agents can be administered at lower (e.g., sub-therapeutic) or higher doses than would otherwise be prescribed. In the event that lower doses are administered, the method of the invention provides that the administration of the lower dose of the anti-microbial agent with the immunostimulatory nucleic acid results in greater than expected therapeutic or prophylactic efficacy. In the event that higher doses of anti-microbial agent are administered, the method provides that the combined administration with an immunostimulatory nucleic acid does not result in as many side effects as are ordinarily observed at those dosage levels. Thus, the various combinations have many advantages over the prior art methods of treating infectious disease.

The immunostimulatory nucleic acids when combined with the anti-microbial agents have many advantages over the use of each composition alone for the treatment of infectious disease. The immunostimulatory nucleic acids function in some aspects by simultaneously inducing innate and antigen specific immune responses leading to a multifaceted attack by the immune system on the microorganism. The anti-microbial agents specifically attack the microorganism, causing death or inhibition of the microorganism. The immunostimulatory nucleic acids provide long-lasting effects, thus reducing dosing regimes, improving compliance and maintenance therapy, reducing emergency situations; and improving quality of life.

Immunostimulatory nucleic acids stimulate the immune system to prevent or treat infectious disease. The strong yet balanced, cellular and humoral immune responses that result from the immune stimulatory capacity of the nucleic acid reflect the natural defense system of the subject against invading microorganisms.

As used herein, the term “prevent”, “prevented”, or “preventing” and “treat”, “treated” or “treating” when used with respect to the prevention or treatment of an infectious disease refers to a prophylactic treatment which increases the resistance of a subject to a microorganism or, in other words, decreases the likelihood that the subject will develop an infectious disease to the microorganism, as well as to a treatment after the subject has been infected in order to fight the infectious disease, e.g., reduce or eliminate it altogether or prevent it from becoming worse.

An “immunostimulatory nucleic acid” as used herein is any nucleic acid containing an immunostimulatory motif or backbone that is capable of inducing an immune response. An induction in an immune response as used herein, refers to any increase in number or activity of an immune cell, or an increase in expression or absolute levels of an immune factor, such as a cytokine. Immune cells include, but are not limited to, NK cells, CD4+ T lymphocytes, CD8+ T lymphocytes, B cells, dendritic cells, macrophage and other antigen-presenting cells. Cytokines include, but are not limited to, interleukins, TNF-α, IFN-α, β, and γ, Flt-ligand, and co-stimulatory molecules. Immunostimulatory motifs include, but are not limited to, CpG motifs, poly-G motifs, and T-rich motifs. Immunostimulatory backbones include, but are not limited to, phosphate modified backbones, such as phosphorothioate backbones. Immunostimulatory nucleic acids have been described extensively in the prior art and a brief summary of these nucleic acids is presented below.

The terms “nucleic acid” and “oligonucleotide” are used interchangeably to mean multiple nucleotides (i.e. molecules comprising a sugar (e.g. ribose or deoxyribose) linked to a phosphate group and to an exchangeable organic base, which is either a substituted pyrimidine (e.g. cytosine (C), thymine (T) or uracil (U)) or a substituted purine (e.g. adenine (A) or guanine (G)). As used herein, the terms refer to oligoribonucleotides as well as oligodeoxyribonucleotides. The terms shall also include polynucleosides (i.e. a polynucleotide minus the phosphate) and any other organic base containing polymer. Nucleic acids include vectors, e.g., plasmids as well as oligonucleotides. Nucleic acid molecules can be obtained from existing nucleic acid sources (e.g. genomic or cDNA), but are preferably synthetic (e.g. produced by oligonucleotide synthesis). When the immunostimulatory nucleic acid is in the form of a vector, it is not the same vector that is used to express the peptide anti-microbial agent, unless more than one anti-microbial agent is used in the method or is present in the composition. In some embodiments, the immunostimulatory nucleic acid is not in the form of an expression vector. In other embodiments the immunostimulatory nucleic acid is not an antisense oligonucleotide.

Immunostimulatory nucleic acids may possess immunostimulatory motifs such as unmethylated CpG motifs, methylated CpG motifs, and non-CpG motifs such as poly-G motifs, and T-rich motifs. Depending upon the embodiment of the invention, some immunostimulatory motifs are preferred over others. In some embodiments, any nucleic acid, regardless of whether it possesses an identifiable motif, can be used in the combination therapy. Immunostimulatory nucleic acids also include nucleic acids having a modified backbone, such as a phosphorothioate modified backbone. In particular embodiments, the immunostimulatory nucleic acids having a phosphorothioate modified backbone does not also have an identifiable motif, yet it is still immunostimulatory. Some aspects of the invention, particularly those directed at treating a subject having or at risk of developing an infectious disease, do not embrace the use of T-rich or methylated CpG nucleic acids (i.e., nucleic acids that possess either a T-rich or a methylated CpG motif). A methylated CpG nucleic acid as used herein refers to a nucleic acid having a CpG dinucleotide in which the C residue is methylated.

In some embodiments, the immunostimulatory nucleic acid is a CpG nucleic acid. CpG sequences, while relatively rare in human DNA are commonly found in the DNA of infectious organisms such as bacteria. The human immune system has apparently evolved to recognize CpG sequences as an early warning sign of infection and to initiate an immediate and powerful immune response against invading pathogens without causing adverse reactions frequently seen with other immune stimulatory agents. Thus CpG containing nucleic acids, relying on this innate immune defense mechanism can utilize a unique and natural pathway for immune therapy. The effects of CpG nucleic acids on immune modulation have been described extensively in U.S. Pat. No. 6,194,388, and published patent applications, such as PCT US95/01570), PCT/US97/19791, PCT/US98/03678; PCT/US98/10408; PCT/US98/04703; PCT/US99/07335; and PCT/US99/09863. The entire contents of each of these issued patents and patent applications are hereby incorporated by reference.

A CpG nucleic acid is a nucleic acid which includes at least one unmethylated CpG dinucleotide. A nucleic acid containing at least one unmethylated CpG dinucleotide is a nucleic acid molecule which contains an unmethylated cytosine in a cytosine-guanine dinucleotide sequence (i.e. “CpG DNA” or DNA containing a 5′ cytosine followed by 3′ guanosine and linked by a phosphate bond) and activates the immune system. The CpG nucleic acids can be double-stranded or single-stranded. Generally, double-stranded molecules are more stable in vivo, while single-stranded molecules have increased immune activity. Thus in some aspects of the invention it is preferred that the nucleic acid be single stranded and in other aspects it is preferred that the nucleic acid be double stranded. The entire immunostimulatory nucleic acid can be unmethylated or portions may be unmethylated but at least the C of the 5′ CG 3′ must be unmethylated.

In one preferred embodiment the invention provides an immunostimulatory nucleic acid which is a CpG nucleic acid represented by at least the formula:

5′X₁X₂CGX₃X₄3′

wherein X₁, X₂,X₃, and X₄ are nucleotides. In one embodiment X₂ is adenine, guanine, cytosine, or thymine. In another embodiment X₃ is cytosine, guanine, adenine, or thymine. In other embodiments X₂ is adenine, guanine, or thymine and X₃ is cytosine, adenine, or thymine.

In another embodiment the immunostimulatory nucleic acid is an isolated CpG nucleic acid represented by at least the formula:

5′N₁X₁X₂CGX₃X₄N₂3′

wherein X₁, X₂, X₃, and X₄ are nucleotides and N is any nucleotide and N₁ and N₂ are nucleic acid sequences composed of from about 0-50 N's each. In one embodiment X₁X₂ are nucleotides selected from the group consisting of: GpT, GpG, GpA, ApA, ApT, ApG, CpT, CpA, CpG, TpA, TpT, and TpG; and X₃X₄ are nucleotides selected from the group consisting of: TpT, ApT, TpG, ApG, CpG, TpC, ApC, CpC, TpA, ApA, and CpA. Preferably X₁X₂ are GpA or GpT and X₃X₄ are TpT. In other embodiments X₁ or X₂ or both are purines and X₃ or X₄ or both are pyrimidines or X₁X₂ are GpA and X₃ or X₄ or both are pyrimidines. In another preferred embodiment X₁X₂ are nucleotides selected from the group consisting of: TpA, ApA, ApC, ApG, and GpG. In yet another embodiment X₃X₄ are nucleotides selected from the group consisting of: TpT, TpA, TpG, ApA, ApG, ApC, and CpA. X₁X₂ in another embodiment are nucleotides selected from the group consisting of: TpT, TpG, ApT, GpC, CpC, CpT, TpC, GpT and CpG.

In another preferred embodiment the immunostimulatory nucleic acid has the sequence 5′TCN₁TX₁X₂CGX₃X₄3′, wherein X₁, X₂, X₃, and X₄ are nucleotides and N is as described above. The immunostimulatory nucleic acids of the invention in some embodiments include X₁X₂ selected from the group consisting of GpT, GpG, GpA and ApA and X₃X₄ is selected from the group consisting of TpT, CpT and TpC.

For facilitating uptake into cells, the immunostimulatory nucleic acids are preferably in the range of 6 to 100 bases in length. However, nucleic acids of any size greater than 6 nucleotides (even many kb long) are capable of inducing an immune response according to the invention if sufficient immunostimulatory motifs are present. Preferably the immunostimulatory nucleic acid is in the range of between 8 and 100 and in some embodiments between 8 and 50 or 8 and 30 nucleotides in size.

“Palindromic sequence” shall mean an inverted repeat (i.e. a sequence such as ABCDEE′D′C′B′A′ in which A and A′ are bases capable of forming the usual Watson-Crick base pairs. In vivo, such sequences may form double-stranded structures. In one embodiment the CpG nucleic acid contains a palindromic sequence. A palindromic sequence used in this context refers to a palindrome in which the CpG is part of the palindrome, and preferably is the center of the palindrome. In another embodiment the CpG nucleic acid is free of a palindrome. An immunostimulatory nucleic acid that is free of a palindrome is one in which the CpG dinucleotide is not part of a palindrome. Such an oligonucleotide may include a palindrome in which the CpG is not the center of the palindrome.

In some embodiments of the invention, a non-CpG immunostimulatory nucleic acid is used. A non-CpG immunostimulatory nucleic acid is a nucleic acid that does not have a CpG motif in its sequence, regardless of whether the C residue of the dinucleotide is methylated or unmethylated. Non-CpG immunostimulatory nucleic acids may induce Th1 or Th2 immune responses, depending upon their sequence, their mode of delivery, and the dose at which they are administered.

One important category of non-CpG nucleic acids are poly-G nucleic acids. Poly-G nucleic acids are also immunostimulatory, and are useful in some aspects of the invention. A variety of references, including Pisetsky and Reich, 1993 Mol. Biol. Reports, 18:217-221; Krieger and Herz, 1994, Ann. Rev. Biochem., 63:601-637; Macaya et al., 1993, PNAS, 90:3745-3749; Wyatt et al., 1994, PNAS, 91:1356-1360; Rando and Hogan, 1998, In Applied Antisense Oligonucleotide Technology, ed. Krieg and Stein, p. 335-352; and Kimura et al., 1994, J. Biochem. 116, 991-994 also describe the immunostimulatory properties of poly-G nucleic acids. In accordance with one aspect of the invention, poly-G-containing nucleotides are useful, inter alia, for treating and preventing bacterial and viral infections.

Poly-G nucleic acids preferably are nucleic acids having the following formulas:

5′ X₁X₂GGGX₃X₄ 3′

wherein X₁, X₂, X₃, and X₄ are nucleotides. In preferred embodiments at least one of X₃ and X₄ are a G. In other embodiments both of X₃ and X₄ are a G. In yet other embodiments the preferred formula is 5′ GGGNGGG3′, or 5′ GGGNGGGNGGG3′ (SEQ ID NO:134) wherein N represents between 0 and 20 nucleotides. In other embodiments the Poly-G nucleic acid is free of unmethylated CG dinucleotides, such as, for example, the nucleic acids listed above as SEQ ID NO 95-133. In other embodiments the Poly-G nucleic acid includes at least one unmethylated CG dinucleotide, such as, for example, the nucleic acids listed above as SEQ ID NO 46, 47, 58, and 61.

In select aspects of the invention, the non-CpG immunostimulatory nucleic acids may be T-rich nucleic acids. T-rich nucleic acids are nucleic acids having T-rich motifs. T rich motifs and nucleic acids possessing such motifs are described in U.S. patent application Ser. No. 09/669,187, filed Sep. 25, 2000, by Krieg et al., the entire contents of which are incorporated herein by reference. Other non-CpG nucleic acids useful in the present invention are described in U.S. patent application Ser. No. 09/768,012, filed Jan. 22, 2001, the entire contents of which are incorporated herein in their entirety by reference.

Exemplary immunostimulatory nucleic acid sequences include but are not limited to those immunostimulatory sequences shown in Table 1.

TABLE 1

| | |
|---|---|
| GCTAGACGTTAGCGT; | (SEQ ID NO: 1) |
| GCTAGATGTTAGCGT; | (SEQ ID NO: 2) |
| GCTAGACGTTAGCGT; | (SEQ ID NO: 3) |
| GCTAGACGTTAGCGT; | (SEQ ID NO: 4) |
| GCATGACGTTGAGCT; | (SEQ ID NO: 5) |
| ATGGAAGGTCCAGCGTTCTC; | (SEQ ID NO: 6) |
| ATCGACTCTCGAGCGTTCTC; | (SEQ ID NO: 7) |
| ATCGACTCTCGAGCGTTCTC; | (SEQ ID NO: 8) |
| ATCGACTCTCGAGCGTTCTC; | (SEQ ID NO: 9) |
| ATGGAAGGTCCAACGTTCTC; | (SEQ ID NO: 10) |
| GAGAACGCTGGACCTTCCAT; | (SEQ ID NO: 11) |
| GAGAACGCTCGACCTTCCAT; | (SEQ ID NO: 12) |
| GAGAACGCTCGACCTTCGAT; | (SEQ ID NO: 13) |
| GAGAACGCTGGACCTTCCAT; | (SEQ ID NO: 14) |
| GAGAACGATGGACCTTCCAT; | (SEQ ID NO: 15) |
| GAGAACGCTCCAGCACTGAT; | (SEQ ID NO: 16) |
| TCCATGTCGGTCCTGATGCT; | (SEQ ID NO: 17) |
| TCCATGTCGGTCCTGATGCT; | (SEQ ID NO: 18) |
| TCCATGACGTTCCTGATGCT; | (SEQ ID NO: 19) |
| TCCATGTCGGTCCTGCTGAT; | (SEQ ID NO: 20) |
| TCAACGTT; | (SEQ ID NO: 21) |
| TCAGCGCT; | (SEQ ID NO: 22) |
| TCATCGAT; | (SEQ ID NO: 23) |
| TCTTCGAA; | (SEQ ID NO: 24) |
| CAACGTT; | (SEQ ID NO: 25) |
| CCAACGTT; | (SEQ ID NO: 26) |
| AACGTTCT; | (SEQ ID NO: 27) |
| TCAACGTC; | (SEQ ID NO: 28) |
| ATGGACTCTCCAGCGTTCTC; | (SEQ ID NO: 29) |
| ATGGAAGGTCCAACGTTCTC; | (SEQ ID NO: 30) |
| ATCGACTCTCGAGCGTTCTC; | (SEQ ID NO: 31) |
| ATGGAGGCTCCATCGTTCTC; | (SEQ ID NO: 32) |
| ATCGACTCTCGAGCGTTCTC; | (SEQ ID NO: 33) |
| ATCGACTCTCGAGCGTTCTC; | (SEQ ID NO: 34) |
| TCCATGTCGGTCCTGATGCT; | (SEQ ID NO: 35) |
| TCCATGCCGGTCCTGATGCT; | (SEQ ID NO: 36) |
| TCCATGGCGGTCCTGATGCT; | (SEQ ID NO: 37) |
| TCCATGACGGTCCTGATGCT; | (SEQ ID NO: 38) |
| TCCATGTCGATCCTGATGCT; | (SEQ ID NO: 39) |
| TCCATGTCGCTCCTGATGCT; | (SEQ ID NO: 40) |
| TCCATGTCGTCCCTGATGCT; | (SEQ ID NO: 41) |
| TCCATGACGTGCCTGATGCT; | (SEQ ID NO: 42) |
| TCCATAACGTTCCTGATGCT; | (SEQ ID NO: 43) |
| TCCATGACGTCCCTGATGCT; | (SEQ ID NO: 44) |
| TCCATCACGTGCCTGATGCT; | (SEQ ID NO: 45) |
| GGGGTCAACGTTGACGGGG; | (SEQ ID NO: 46) |
| GGGGTCAGTCGTGACGGGG; | (SEQ ID NO: 47) |
| GCTAGACGTTAGTGT; | (SEQ ID NO: 48) |
| TCCATGTCGTTCCTGATGCT; | (SEQ ID NO: 49) |
| ACCATGGACGATCTGTTTCCCCTC; | (SEQ ID NO: 50) |
| TCTCCCAGCGTGCGCCAT; | (SEQ ID NO: 51) |
| ACCATGGACGAACTGTTTCCCCTC; | (SEQ ID NO: 52) |
| ACCATGGACGAGCTGTTTCCCCTC; | (SEQ ID NO: 53) |
| ACCATGGACGACCTGTTTCCCCTC; | (SEQ ID NO: 54) |
| ACCATGGACGTACTGTTTCCCCTC; | (SEQ ID NO: 55) |
| ACCATGGACGGTCTGTTTCCCCTC; | (SEQ ID NO: 56) |
| ACCATGGACGTTCTGTTTCCCCTC; | (SEQ ID NO: 57) |
| CACGTTGAGGGGCAT; | (SEQ ID NO: 58) |
| TCAGCGTGCGCC; | (SEQ ID NO: 59) |
| ATGACGTTCCTGACGTT; | (SEQ ID NO: 60) |
| TCTCCCAGCGGGCGCAT; | (SEQ ID NO: 61) |
| TCCATGTCGTTCCTGTCGTT; | (SEQ ID NO: 62) |
| TCCATAGCGTTCCTAGCGTT; | (SEQ ID NO: 63) |
| TCGTCGCTGTCTCCCCTTCTT; | (SEQ ID NO: 64) |
| TCCTGACGTTCCTGACGTT; | (SEQ ID NO: 65) |
| TCCTGTCGTTCCTGTCGTT; | (SEQ ID NO: 66) |
| TCCATGTCGTTTTTGTCGTT; | (SEQ ID NO: 67) |
| TCCTGTCGTTCCTTGTCGTT; | (SEQ ID NO: 68) |
| TCCTTGTCGTTCCTGTCGTT; | (SEQ ID NO: 69) |
| TCCTGTCGTTTTTTGTCGTT; | (SEQ ID NO: 70) |
| TCGTCGCTGTCTGCCCTTCTT; | (SEQ ID NO: 71) |
| TCGTCGCTGTTGTCGTTTCTT; | (SEQ ID NO: 72) |
| TCCATGCGTGCGTGCGTTTT; | (SEQ ID NO: 73) |
| TCCATGCGTTGCGTTGCGTT; | (SEQ ID NO: 74) |
| TCCACGACGTTTTCGACGTT; | (SEQ ID NO: 75) |
| TCGTCGTTGTCGTTGTCGTT; | (SEQ ID NO: 76) |
| TCGTCGTTTTGTCGTTTTGTCGTT; | (SEQ ID NO: 77) |
| TCGTCGTTGTCGTTTTGTCGTT; | (SEQ ID NO: 78) |
| GCGTGCGTTGTCGTTGTCGTT; | (SEQ ID NO: 79) |
| TGTCGTTTGTCGTTTGTCGTT; | (SEQ ID NO: 80) |
| TGTCGTTGTCGTTGTCGTTGTCGTT; | (SEQ ID NO: 81) |
| TGTCGTTGTCGTTGTCGTT; | (SEQ ID NO: 82) |
| TCGTCGTCGTCGTT; | (SEQ ID NO: 83) |
| TGTCGTTGTCGTT; | (SEQ ID NO: 84) |
| TCCATAGCGTTCCTAGCGTT; | (SEQ ID NO: 85) |
| TCCATGACGTTCCTGACGTT; | (SEQ ID NO: 86) |
| GTCGYT; | (SEQ ID NO: 87) |
| TGTCGYT; | (SEQ ID NO: 88) |
| AGCTATGACGTTCCAAGG; | (SEQ ID NO: 89) |
| TCCATGACGTTCCTGACGTT; | (SEQ ID NO: 90) |
| ATCGACTCTCGAACGTTCTC; | (SEQ ID NO: 91) |
| TCCATGTCGGTCCTGACGCA; | (SEQ ID NO: 92) |
| TCTTCGAT; | (SEQ ID NO: 93) |
| ATAGGAGGTCCAACGTTCTC; | (SEQ ID NO: 94) |
| GCTAGAGGGGAGGGT; | (SEQ ID NO: 95) |
| GCTAGATGTTAGGGG; | (SEQ ID NO: 96) |
| GCTAGAGGGGAGGGT; | (SEQ ID NO: 97) |
| GCTAGAGGGGAGGGT; | (SEQ ID NO: 98) |
| GCATGAGGGGGAGCT; | (SEQ ID NO: 99) |
| ATGGAAGGTCCAGGGGGCTC; | (SEQ ID NO: 100) |
| ATGGACTCTGGAGGGGGCTC; | (SEQ ID NO: 101) |
| ATGGACTCTGGAGGGGGCTC; | (SEQ ID NO: 102) |
| ATGGACTCTGGAGGGGGCTC; | (SEQ ID NO: 103) |
| ATGGAAGGTCCAAGGGGCTC; | (SEQ ID NO: 104) |
| GAGAAGGGGGGACCTTCCAT; | (SEQ ID NO: 105) |
| GAGAAGGGGGGACCTTCCAT; | (SEQ ID NO: 106) |
| GAGAAGGGGGGACCTTGGAT; | (SEQ ID NO: 107) |
| GAGAAGGGGGGACCTTCCAT; | (SEQ ID NO: 108) |
| GAGAAGGGGGGACCTTCCAT; | (SEQ ID NO: 109) |
| GAGAAGGGGCCAGCACTGAT; | (SEQ ID NO: 110) |
| TCCATGTGGGGCCTGATGCT; | (SEQ ID NO: 111) |
| TCCATGTGGGGCCTGATGCT; | (SEQ ID NO: 112) |
| TCCATGAGGGGCCTGATGCT; | (SEQ ID NO: 113) |
| TCCATGTGGGGCCTGCTGAT; | (SEQ ID NO: 114) |
| ATGGACTCTCCGGGGTTCTC; | (SEQ ID NO: 115) |
| ATGGAAGGTCCGGGGTTCTC; | (SEQ ID NO: 116) |
| ATGGACTCTGGAGGGGTCTC; | (SEQ ID NO: 117) |
| ATGGAGGCTCCATGGGGCTC; | (SEQ ID NO: 118) |
| ATGGACTCTGGGGGGTTCTC; | (SEQ ID NO: 119) |
| ATGGACTCTGGGGGGTTCTC; | (SEQ ID NO: 120) |
| TCCATGTGGGTGGGGATGCT; | (SEQ ID NO: 121) |
| TCCATGCGGGTGGGGATGCT; | (SEQ ID NO: 122) |
| TCCATGGGGGTCCTGATGCT; | (SEQ ID NO: 123) |
| TCCATGGGGGTCCTGATGCT; | (SEQ ID NO: 124) |
| TCCATGTGGGGCCTGATGCT; | (SEQ ID NO: 125) |
| TCCATGTGGGGCCTGATGCT; | (SEQ ID NO: 126) |
| TCCATGGGGTCCCTGATGCT; | (SEQ ID NO: 127) |
| TCCATGGGGTGCCTGATGCT; | (SEQ ID NO: 128) |
| TCCATGGGGTTCCTGATGCT; | (SEQ ID NO: 129) |
| TCCATGGGGTCCCTGATGCT; | (SEQ ID NO: 130) |
| TCCATCGGGGGCCTGATGCT; | (SEQ ID NO: 131) |
| GCTAGAGGGAGTGT; | (SEQ ID NO: 132) |
| GGGGGGGGGGGGGGGGGGGG; | (SEQ ID NO: 133) |

Nucleic acids having modified backbones, such as phosphorothioate backbones, fall within the class of non-CpG immunostimulatory nucleic acids. U.S. Pat. Nos. 5,723,335 and 5,663,153 issued to Hutcherson, et al. and related PCT publication WO95/26204 describe immune stimulation using phosphorothioate oligonucleotide analogues. These patents describe the ability of the phosphorothioate backbone to stimulate an immune response in a non-sequence specific manner. Thus, some embodiments of the invention rely on the use of phosphorothioate backbone nucleic acids that lack methylated and unmethylated CpG, poly-G and T-rich motifs.

In the case when the immunostimulatory nucleic acid is administered in conjunction with a nucleic acid vector, it is preferred that the backbone of the immunostimulatory nucleic acid be a chimeric combination of phosphodiester and phosphorothioate (or other phosphate modification). The cell may have a problem taking up a plasmid vector in the presence of completely phosphorothioate oligonucleotide. Thus when both a vector and an oligonucleotide are delivered to a subject, it is preferred that the oligonucleotide have a chimeric backbone or have a phosphorothioate backbone but that the plasmid is associated with a vehicle that delivers it directly into the cell, thus avoiding the need for cellular uptake. Such vehicles are known in the art and include, for example, liposomes and gene guns.

For use in the instant invention, the immunostimulatory nucleic acids can be synthesized de novo using any of a number of procedures well known in the art. Such compounds are referred to as “synthetic nucleic acids.” For example, the b-cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et al., Nucl. Acid. Res. 14:5399-5407, 1986; Garegg et al., Tet. Let. 27:4055-4058, 1986, Gaffney et al., Tet. Let. 29:2619-2622, 1988). These chemistries can be performed by a variety of automated oligonucleotide synthesizers available in the market. These nucleic acids are referred to as synthetic nucleic acids. Alternatively, immunostimulatory nucleic acids can be produced on a large scale in plasmids, (see Sambrook, T., et al., “Molecular Cloning: A Laboratory Manual”, Cold Spring Harbor laboratory Press, New York, 1989) and separated into smaller pieces or administered whole. Nucleic acids can be prepared from naturally occurring nucleic acid sequences (e.g., genomic DNA or cDNA) using known techniques, such as those employing restriction enzymes, exonucleases or endonucleases. Nucleic acids prepared in this manner are referred to as “isolated nucleic acids.” The term “immunostimulatory nucleic acid” encompasses both synthetic and isolated immunostimulatory nucleic acids.

For use in vivo, nucleic acids are preferably relatively resistant to degradation (e.g., are stabilized). A “stabilized nucleic acid molecule” shall mean a nucleic acid molecule that is relatively resistant to in vivo degradation (e.g. via an exo- or endo-nuclease). Stabilization can be a function of length, secondary structure, backbone, etc. Immunostimulatory nucleic acids that are tens to hundreds of kbs long are relatively resistant to in vivo degradation. For shorter immunostimulatory nucleic acids, secondary structure can stabilize and increase their effect. For example, if the 3′ end of a nucleic acid has self-complementarity to an upstream region, so that it can fold back and form a sort of stem loop structure, then the nucleic acid becomes stabilized and therefore exhibits more activity.

Alternatively, nucleic acid stabilization can be accomplished via backbone modifications. Preferred stabilized nucleic acids of the instant invention have a modified backbone. It has been demonstrated that modification of the nucleic acid backbone provides enhanced activity of the immunostimulatory nucleic acids when administered in vivo. One type of modified backbone is a phosphate backbone modification. Immunostimulatory nucleic acids, including at least two phosphorothioate linkages at the 5′ end of the oligonucleotide and multiple phosphorothioate linkages at the 3′ end, preferably 5, can in some circumstances provide maximal activity and protect the nucleic acid from degradation by intracellular exo- and endo-nucleases. Other phosphate modified nucleic acids include phosphodiester modified nucleic acids, combinations of phosphodiester and phosphorothioate nucleic acids, methylphosphonate, methylphosphorothioate, phosphorodithioate, and combinations thereof. Each of these combinations in CpG nucleic acids and their particular effects on immune cells is discussed in more detail in PCT Published Patent Applications PCT/US95/01570 and PCT/US97/19791, the entire contents of which are hereby incorporated by reference. Although Applicants are not bound by the theory, it is believed that these phosphate modified nucleic acids may show more stimulatory activity due to enhanced nuclease resistance, increased cellular uptake, increased protein binding, and/or altered intracellular localization.

Modified backbones such as phosphorothioates may be synthesized using automated techniques employing either phosphoramidate or H-phosphonate chemistries. Aryl- and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann, E. and Peyman, A., Chem. Rev. 90:544, 1990; Goodchild, J., Bioconjugate Chem. 1:65, 1990).

Both stabilized nucleic acids and phosphodiester nucleic acids containing immunostimulatory motifs are active in immune cells. However, based on the concentration needed to induce immunostimulatory nucleic acid specific effects, the nuclease resistant nucleic acids are more potent, and in some cases can be used in lower doses. This depends, of course, on the mode of delivery, formulation, etc. For instance, lower doses of phosphodiester nucleic acids are not required if the nucleic acid is delivered directly to the cell, e.g. using gene gun or liposomes.

Another type of modified backbone, useful according to the invention, is a peptide-nucleic acid. The backbone is composed of aminoethylglycine which provides the DNA-character. The backbone does not include any phosphate and thus may optionally have no net charge. The lack of charge allows for stronger DNA-DNA binding because the charge repulsion between the two strands does not exist. Additionally, because the backbone has an extra methylene group, the oligonucleotides are enzyme/protease resistant. Peptide-nucleic acids can be purchased from various commercial sources, e.g., Perkin Elmer, C. A. or synthesized de novo.

Another class of backbone modifications include 2′-O-methylribonucleosides (2′-Ome). These types of substitutions are described extensively in the prior art and in particular with respect to their immunostimulating properties in Zhao et al., Bioorganic and Medicinal Chemistry Letters, 1999, 9:24:3453. Zhao et al. describes methods of preparing 2′-Ome modifications to nucleic acids.

The nucleic acid molecules of the invention may include naturally-occurring or synthetic purine or pyrimidine heterocyclic bases as well as modified backbones. Purine or pyrimidine heterocyclic bases include, but are not limited to, adenine, guanine, cytosine, thymidine, uracil, and inosine. Other representative heterocyclic bases are disclosed in U.S. Pat. No. 3,687,808, issued to Merigan, et al. and in many other references, well known in the art. The terms purine, pyrimidine, bases, or nucleotides are used herein to refer to both naturally-occurring or synthetic purines, pyrimidines, bases, or nucleotides.

Other stabilized nucleic acids include: nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Nucleic acids which contain diol, such as tetraethyleneglycol or hexaethyleneglycol, at either or both termini have also been shown to be substantially resistant to nuclease degradation.

The immunostimulatory nucleic acids having backbone modifications useful according to the invention in some embodiments are S- or R-chiral immunostimulatory nucleic acids. An “S chiral immunostimulatory nucleic acid” as used herein is an immunostimulatory nucleic acid wherein at least two nucleotides have a backbone modification forming a chiral center and wherein a plurality of the chiral centers have S chirality. An “R chiral immunostimulatory nucleic acid” as used herein is an immunostimulatory nucleic acid wherein at least two nucleotides have a backbone modification forming a chiral center and wherein a plurality of the chiral centers have R chirality. The backbone modification may be any type of modification that forms a chiral center. The modifications include but are not limited to phosphorothioate, methylphosphonate, methylphosphorothioate, phosphorodithioate, 2′-Ome and combinations thereof.

The chiral immunostimulatory nucleic acids must have at least two nucleotides within the nucleic acid that have a backbone modification. All or less than all of the nucleotides in the nucleic acid, however, may have a modified backbone. Of the nucleotides having a modified backbone (referred to as chiral centers), a plurality have a single chirality, S or R. A “plurality” as used herein refers to an amount greater than 50%. Thus, less than all of the chiral centers may have S or R chirality as long as a plurality of the chiral centers have S or R chirality. In some embodiments at least 55%, 60%, 65%, 70%, 75%, 80%, 85%, 90%, 95%, or 100% of the chiral centers have S or R chirality. In other embodiments at least 55%, 60%, 65%, 70%, 75%, 80%, 85%, 90%, 95%, or 100% of the nucleotides have backbone modifications.

The S- and R-chiral immunostimulatory nucleic acids may be prepared by any method known in the art for producing chirally pure oligonucleotides. The Stec et al reference teaches methods for producing stereopure phosphorothioate oligodeoxynucleotides using an oxathiaphospholane. (Stec, W. J., et al., 1995, J. Am. Chem. Soc., 117:12019). Other methods for making chirally pure oligonucleotides have been described by companies such as ISIS Pharmaceuticals. US Patents have also described these methods. For instance U.S. Pat. Nos. 5,883,237; 5,837,856; 5,599,797; 5,512,668; 5,856,465; 5,359,052; 5,506,212; 5,521,302; and 5,212,295, each of which is hereby incorporated by reference in its entirety, disclose methods for generating stereopure oligonucleotides.

The immunostimulatory nucleic acids are useful for treating or preventing infectious disease in a subject. A “subject” shall mean a human or vertebrate mammal including but not limited to a dog, cat, horse, cow, pig, sheep, goat, or primate, e.g., monkey.

The immunostimulatory nucleic acids are useful in some aspects of the invention as a prophylactic for the treatment of a subject at risk of developing an infectious disease where the exposure of the subject to a microorganism or expected exposure to a microorganism is known or suspected. A “subject at risk” of developing an infectious disease as used herein is a subject who has any risk of exposure to a microorganism, e.g. someone who is in contact with an infected subject or who is traveling to a place where a particular microorganism is found. For instance, a subject at risk may be a subject who is planning to travel to an area where a particular microorganism is found or it may even be any subject living in an area where a microorganism has been identified. A subject at risk of developing an infectious disease includes those subjects that have a general risk of exposure to a microorganism, e.g., influenza, but that don't have the active disease during the treatment of the invention as well as subjects that are considered to be at specific risk of developing an infectious disease because of medical or environmental factors, that expose them to a particular microorganism.

In addition to the use of the immunostimulatory nucleic acid and the anti-microbial agent for prophylactic treatment, the invention also encompasses the use of the combination of drugs for the treatment of a subject having an infectious disease. A “subject having an infectious disease” is a subject that has had contact with a microorganism. Thus the microorganism has invaded the body of the subject. The word “invade” as used herein refers to contact by the microorganism with the external surface of the subject, e.g., skin or mucosal membranes and/or refers to the penetration of the external surface of the subject by the microorganism.

An “infectious disease” as used herein, refers to a disorder arising from the invasion of a host, superficially, locally, or systemically, by an infectious microorganism. Infectious microorganisms include bacteria, viruses, and fungi. Bacteria are unicellular organisms which multiply asexually by binary fission. They are classified and named based on their morphology, staining reactions, nutrition and metabolic requirements, antigenic structure, chemical composition, and genetic homology. Bacteria can be classified into three groups based on their morphological forms, spherical (coccus), straight-rod (bacillus) and curved or spiral rod (vibrio, campylobacter, spirillum, and spirochaete). Bacteria are also more commonly characterized based on their staining reactions into two classes of organisms, gram-positive and gram-negative. Gram refers to the method of staining which is commonly performed in microbiology labs. Gram-positive organisms retain the stain following the staining procedure and appear a deep violet color. Gram-negative organisms do not retain the stain but take up the counter-stain and thus appear pink.

Bacteria have two main structural components, a rigid cell wall and protoplast (material enclosed by the cell wall). The protoplast includes cytoplasm and genetic material. Surrounding the protoplast is the cytoplasmic membrane which includes some of the cell respiratory enzymes and is responsible for the permeability of bacteria and transport of many small molecular weight substances. The cell wall surrounding the cytoplasmic membrane and protoplast is composed of mucopeptides which include complex polymers of sugars cross-linked by peptide chains of amino acids. The wall is also composed of polysaccharides and teichoic acids.

Infectious bacteria include, but are not limited to, gram negative and gram positive bacteria. Gram positive bacteria include, but are not limited to Pasteurella species, Staphylococci species, and Streptococcus species. Gram negative bacteria include, but are not limited to, Escherichia coli, Pseudomonas species, and Salmonella species. Specific examples of infectious bacteria include but are not limited to: Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic species.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Viruses are small infectious agents which contain a nucleic acid core and a protein coat, but are not independently living organisms. A virus cannot survive in the absence of a living cell within which it can replicate. Viruses enter specific living cells either by endocytosis or direct injection of DNA (phage) and multiply, causing disease. The multiplied virus can then be released and infect additional cells. Some viruses are DNA-containing viruses and other are RNA-containing viruses.

Once the virus enters the cell it can cause a variety of physiological effects. One effect is cell degeneration, in which the accumulation of virus within the cell causes the cell to die and break into pieces and release the virus. Another effect is cell fusion, in which infected cells fuse with neighboring cells to produce syncytia. Other types of virus cause cell proliferation which results in tumor formation.

Viruses include, but are not limited to, interoviruses (including, but not limited to, viruses that the family picornaviridae, such as polio virus, coxsackie virus, echo virus), rotaviruses, adenovirus, hepatitus. Specific examples of viruses that have been found in humans include but are not limited to: Retroviridae (e.g. human immunodeficiency viruses, such as HIV-1 (also referred to as HTLV-III, LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class I=internally transmitted; class 2=parenterally transmitted (i.e. Hepatitis C); Norwalk and related viruses, and astroviruses).

In addition to viruses that infect human subjects causing human disorders, the invention is also useful for treating other non-human vertebrates. Non-human vertebrates are also capable of developing infections which can be prevented or treated with the combinations of immunostimulatory nucleic acids and anti-microbials disclosed herein. For instance, in addition to the treatment of infectious human diseases, the methods of the invention are useful for treating or preventing infections of non-human animals.

Infectious virus of both human and non-human vertebrates, include retroviruses, RNA viruses and DNA viruses. This group of retroviruses includes both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of other RNA viruses that are antigens in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that infect vertebrate animals include, but are not limited to: the family Poxviridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A,B,C,D,E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families such as Kuru and Creutzfeldt-Jacob disease viruses and chronic infectious neuropathic agents (CHINA virus).

Fungi are eukaryotic organisms, only a few of which cause infection in vertebrate mammals. Because fungi are eukaryotic organisms, they differ significantly from prokaryotic bacteria in size, structural organization, life cycle and mechanism of multiplication. Fungi are classified generally based on morphological features, modes of reproduction and culture characteristics. Although fungi can cause different types of disease in subjects, such as respiratory allergies following inhalation of fungal antigens, fungal intoxication due to ingestion of toxic substances, such as amatatoxin and phallotoxin produced by poisonous mushrooms and aflotoxins, produced by aspergillus species, not all fungi cause infectious disease.

Infectious fungi can cause systemic or superficial infections. Primary systemic infection can occur in normal healthy subjects and opportunistic infections, are most frequently found in immuno-compromised subjects. The most common fungal agents causing primary systemic infection include blastomyces, coccidioides, and histoplasma. Common fungi causing opportunistic infection in immuno-compromised or immunosuppressed subjects include, but are not limited to, candida albicans (an organism which is normally part of the respiratory tract flora), cryptococcus neoformans (sometimes in normal flora of respiratory tract), and various aspergillus species. Systemic fungal infections are invasive infections of the internal organs. The organism usually enters the body through the lungs, gastrointestinal tract, or intravenous lines. These types of infections can be caused by primary pathogenic fungi or opportunistic fungi.

Superficial fungal infections involve growth of fungi on an external surface without invasion of internal tissues. Typical superficial fungal infections include cutaneous fungal infections involving skin, hair, or nails. An example of a cutaneous infection is Tinea infections, such as ringworm, caused by dermatophytes, such as microsporum or traicophyton species, i.e., microsporum canis, microsporum gypsum, tricofitin rubrum. Examples of fungi include: Cryptococcus neoformans, Histoplasma capsulatum, Coccidioides immitis, Blastomyces dermatitidis, Chlamydia trachomatis, Candida albicans.

Parasitic infections targeted by the methods of the invention include those caused by the following parasites Plasmodium falciparum, Plasmodium ovale, Plasmodium malariae, Plasmdodium vivax, Plasmodium knowlesi, Babesia microti, Babesia divergens, Trypanosoma cruzi, Toxoplasma gondii, Trichinella spiralis, Leishmania major, Leishmania donovani, Leishmania braziliensis and Leishmania tropica, Trypanosoma gambiense, Trypanosmoma rhodesiense and Schistosoma mansoni. In preferred embodiments, the method is directed towards the prevention of infection with parasites which cause malaria.

Other medically relevant microorganisms have been described extensively in the literature, e.g., see C. G. A Thomas, Medical Microbiology, Bailliere Tindall, Great Britain 1983, the entire contents of which is hereby incorporated by reference. Each of the foregoing lists is illustrative, and is not intended to be limiting.

The methods of the invention involve combinations of immunostimulatory nucleic acids and anti-microbial agents for the treatment or prevention of infectious disease. An anti-microbial agent, as used herein, refers to a naturally-occurring or synthetic compound which is capable of killing or inhibiting infectious microorganisms. The type of anti-microbial agent useful according to the invention will depend upon the type of microorganism with which the subject is infected or at risk of becoming infected. In important embodiments, the anti-microbial agent is not conjugated to the immunostimulatory nucleic acid. One type of anti-microbial agent is an antibacterial agent. Antibacterial agents kill or inhibit the growth or function of bacteria. A large class of antibacterial agents is antibiotics.

Antibiotics, which are effective for killing or inhibiting a wide range of bacteria, are referred to as broad spectrum antibiotics. Other types of antibiotics are predominantly effective against the bacteria of the class gram-positive or gram-negative. These types of antibiotics are referred to as narrow spectrum antibiotics. Other antibiotics which are effective against a single organism or disease and not against other types of bacteria, are referred to as limited spectrum antibiotics.

Antibacterial agents are sometimes classified based on their primary mode of action. In general, antibacterial agents are cell wall synthesis inhibitors, cell membrane inhibitors, protein synthesis inhibitors, nucleic acid synthesis or functional inhibitors, and competitive inhibitors. Cell wall synthesis inhibitors inhibit a step in the process of cell wall synthesis, and in general in the synthesis of bacterial peptidoglycan. Cell wall synthesis inhibitors include β-lactam antibiotics, natural penicillins, semi-synthetic penicillins, ampicillin, clavulanic acid, cephalolsporins, and bacitracin.

The β-lactams are antibiotics containing a four-membered β-lactam ring which inhibits the last step of peptidoglycan synthesis. β-lactam antibiotics can be synthesized or natural. The natural antibiotics are generally produced by two groups of fungi, penicillium and cephalosporium molds. The β-lactam antibiotics produced by penicillium are the natural penicillins, such as penicillin G or penicillin V. These are produced by fermentation of penicillium chrysogenum. The natural penicillins have a narrow spectrum of activity and are generally effective against streptococcus, gonococcus, and staphylococcus. Other types of natural penicillins, which are also effective against gram-positive bacteria, include penicillins F, X, K, and O.

Semi-synthetic penicillins are generally modifications of the molecule 6-aminopenicillanic acid produced by a mold. The 6-aminopenicillanic acid can be modified by addition of side chains which produce penicillins having broader spectrums of activity than natural penicillins or various other advantageous properties. Some types of semi-synthetic penicillins have broad spectrums against gram-positive and gram-negative bacteria, but are inactivated by penicillinase. These semi-synthetic penicillins include ampicillin, carbenicillin, oxacillin, azlocillin, mezlocillin, and piperacillin. Other types of semi-synthetic penicillins have narrower activities against gram-positive bacteria, but have developed properties such that they are not inactivated by penicillinase. These include, for instance, methicillin, dicloxacillin, and nafcillin. Some of the broad spectrum semi-synthetic penicillins can be used in combination with β-lactamase inhibitors, such as clavulamic acids and sulbactam. The β-lactamase inhibitors do not have anti-microbial action but they function to inhibit penicillinase, thus protecting the semi-synthetic penicillin from degradation.

One of the serious side effects associated with penicillins, both natural and semi-synthetic, is penicillin-allergy. Penicillin allergies are very serious and can cause death rapidly. In a subject that is allergic to penicillin, the β-lactam molecule will attach to a serum protein which initiates an IgE-mediated inflammatory response. The inflammatory response leads to anaphylaxis and possibly death.

Another type of β-lactam antibiotic is the cephalolsporins. Cephalolsporins are produced by cephalolsporium molds, and have a similar mode of action to penicillin. They are sensitive to degradation by bacterial β-lactamases, and thus, are not always effective alone. Cephalolsporins, however, are resistant to penicillinase. They are effective against a variety of gram-positive and gram-negative bacteria. Cephalolsporins include, but are not limited to, cephalothin, cephapirin, cephalexin, cefamandole, cefaclor, cefazolin, cefuroxine, cefoxitin, cefotaxime, cefsulodin, cefetamet, cefixime, ceftriaxone, cefoperazone, ceftazidine, and moxalactam.

Bacitracin is another class of antibiotics which inhibit cell wall synthesis. These antibiotics, produced by bacillus species, prevent cell wall growth by inhibiting the release of muropeptide subunits or peptidoglycan from the molecule that delivers the subunit to the outside of the membrane. Although bacitracin is effective against gram-positive bacteria, its use is limited in general to topical administration because of its high toxicity. Since lower effective doses of bacitracen can be used when the compound is administered with the immunostimulatory nucleic acids of the invention, this compound can be used systemically and the toxicity reduced.

Carbapenems are another broad spectrum β-lactam antibiotic, which is capable of inhibiting cell wall synthesis. Examples of carbapenems include, but are not limited to, imipenems. Monobactems are also broad spectrum β-lactam antibiotics, and include, euztreonam. An antibiotic produced by streptomyces, vancomycin, is also effective against gram-positive bacteria by inhibiting cell membrane synthesis.

Another class of anti-bacterial agents is the anti-bacterial agents that are cell membrane inhibitors. These compounds disorganize the structure or inhibit the function of bacterial membranes. Alteration of the cytoplasmic membrane of bacteria results in leakage of cellular materials from the cell. Compounds that inhibit or interfere with the cell membrane cause death of the cell because the integrity of the cytoplasmic and outer membranes is vital to bacteria. One problem with anti-bacterial agents that are cell membrane inhibitors is that they can produce effects in eukaryotic cells as well as bacteria because of the similarities in phospholipids in bacterial and eukaryotic membranes. Thus these compounds are rarely specific enough to permit these compounds to be used systemically and prevent the use of high doses for local administration.

One clinically useful anti-bacterial agent that is a cell membrane inhibitor is Polymyxin, produced by Bacillus polymyxis. Polymyxins interfere with membrane function by binding to membrane phospholipids. Polymyxin is effective mainly against Gram-negative bacteria and is generally used in severe Pseudomonas infections or Pseudomonas infections that are resistant to less toxic antibiotics. It is also used in some limited instances topically. The limited use of this agent is due to the severe side effects associated with systemic administration, such as damage to the kidney and other organs.

Other cell membrane inhibitors include Amphotericin B and Nystatin produced by the bacterium Streptomyces which are also anti-fungal agents, used predominantly in the treatment of systemic fungal infections and Candida yeast infections respectively. Imidazoles, produced by the bacterium Streptomyces, are another class of antibiotic that is a cell membrane inhibitor. Imidazoles are used as bacterial agents as well as anti-fungal agents, e.g., used for treatment of yeast infections, dermatophytic infections, and systemic fungal infections. Imidazoles include but are not limited to clotrimazole, miconazole, ketoconazole, itraconazole, and fluconazole.

Many anti-bacterial agents are protein synthesis inhibitors. These compounds prevent bacteria from synthesizing structural proteins and enzymes and thus cause inhibition of bacterial cell growth or function or cell death. In general these compounds interfere with the processes of transcription or translation. Anti-bacterial agents that block transcription include but are not limited to Rifampins, produced by the bacterium Streptomyces and Ethambutol, a synthetic chemical. Rifampins, which inhibit the enzyme RNA polymerase, have a broad spectrum activity and are effective against gram-positive and gram-negative bacteria as well as Mycobacterium tuberculosis. Ethambutol is effective against Mycobacterium tuberculosis.

Anti-bacterial agents which block translation interfere with bacterial ribosomes to prevent mRNA from being translated into proteins. In general this class of compounds includes but is not limited to tetracyclines, chloramphenicol, the macrolides (e.g. erythromycin) and the aminoglycosides (e.g. streptomycin).

Some of these compounds bind irreversibly to the 30s ribosomal subunit and cause a misreading of the mRNA, e.g., the aminoglycosides. The aminoglycosides are a class of antibiotics which are produced by the bacterium Streptomyces, such as, for instance streptomycin, kanamycin, tobramycin, amikacin, and gentamicin. Aminoglycosides have been used against a wide variety of bacterial infections caused by Gram-positive and Gram-negative bacteria. Streptomycin has been used extensively as a primary drug in the treatment of tuberculosis. Gentamicin is used against many strains of Gram-positive and Gram-negative bacteria, including Pseudomonas infections, especially in combination with Tobramycin. Kanamycin is used against many Gram-positive bacteria, including penicillin-resistant staphylococci. One side effect of aminoglycosides that has limited their use clinically is that at dosages which are essential for efficacy, prolonged use has been shown to impair kidney function and cause damage to the auditory nerves leading to deafness.

Another type of translation inhibitor anti-bacterial agent is the tetracyclines. The tetracyclines bind reversibly to the 30s ribosomal subunit and interfere with the binding of charged tRNA to the bacterial ribosome. The tetracyclines are a class of antibiotics, produced by the bacterium Streptomyces, that are broad-spectrum and are effective against a variety of gram-positive and gram-negative bacteria. Examples of tetracyclines include tetracycline, minocycline, doxycycline, and chlortetracycline. They are important for the treatment of many types of bacteria but are particularly important in the treatment of Lyme disease.

As a result of their low toxicity and minimal direct side effects, the tetracyclines have been overused and misused by the medical community, leading to problems. For instance, their overuse has led to wide-spread development of resistance. When used in combination with the immunostimulatory nucleic acids of the invention, these problems can be minimized and tetracyclines can be effectively used for the broad spectrum treatment of many bacteria.

Anti-bacterial agents such as the macrolides bind reversibly to the 50s ribosomal subunit and inhibits elongation of the protein by peptidyl transferase or prevents the release of uncharged tRNA from the bacterial ribosome or both. The macrolides contain large lactone rings linked through glycoside bonds with amino sugars. These compounds include erythromycin, roxithromycin, clarithromycin, oleandomycin, and azithromycin. Erythromycin is active against most Gram-positive bacteria, Neisseria, Legionella and Haemophilus, but not against the Enterobacteriaceae. Lincomycin and clindamycin, which block peptide bond formation during protein synthesis, are used against gram-positive bacteria.

Another type of translation inhibitor is chloramphenicol. Chloramphenicol binds the 70S ribosome inhibiting the bacterial enzyme peptidyl transferase thereby preventing the growth of the polypeptide chain during protein synthesis. Chloramphenicol can be prepared from Streptomyces or produced entirely by chemical synthesis. One serious side effect associated with chloramphenicol is aplastic anemia. Aplastic anemia develops at doses of chloramphenicol which are effective for treating bacteria in a small proportion (1/50,000) of patients. Chloramphenicol which was once a highly prescribed antibiotic is now seldom uses as a result of the deaths from anemia. Because of its effectiveness it is still used in life-threatening situations (e.g. typhoid fever). By combining chloramphenicol with the immunostimulatory nucleic acids these compounds can again be used as anti-bacterial agents because the immunostimulatory agents allow a lower dose of the chloramphenicol to be used, a dose that does not produce side effects.

Some anti-bacterial agents disrupt nucleic acid synthesis or function, e.g., bind to DNA or RNA so that their messages cannot be read. These include but are not limited to quinolones and co-trimoxazole, both synthetic chemicals and rifamycins, a natural or semi-synthetic chemical. The quinolones block bacterial DNA replication by inhibiting the DNA gyrase, the enzyme needed by bacteria to produce their circular DNA. They are broad spectrum and examples include norfloxacin, ciprofloxacin, enoxacin, nalidixic acid and temafloxacin. Nalidixic acid is a bactericidal agent that binds to the DNA gyrase enzyme (topoisomerase) which is essential for DNA replication and allows supercoils to be relaxed and reformed, inhibiting DNA gyrase activity. The main use of nalidixic acid is in treatment of lower urinary tract infections (UTI) because it is effective against several types of Gram-negative bacteria such as E. coli, Enterobacter aerogenes, K. pneumoniae and Proteus species which are common causes of UTI. Co-trimoxazole is a combination of sulfamethoxazole and trimethoprim, which blocks the bacterial synthesis of folic acid needed to make DNA nucleotides. Rifampicin is a derivative of rifamycin that is active against Gram-positive bacteria (including Mycobacterium tuberculosis and meningitis caused by Neisseria meningitidis) and some Gram-negative bacteria. Rifampicin binds to the beta subunit of the polymerase and blocks the addition of the first nucleotide which is necessary to activate the polymerase, thereby blocking mRNA synthesis.

Another class of anti-bacterial agents is compounds that function as competitive inhibitors of bacterial enzymes. The competitive inhibitors are mostly all structurally similar to a bacterial growth factor and compete for binding but do not perform the metabolic function in the cell. These compounds include sulfonamides and chemically modified forms of sulfanilamide which have even higher and broader antibacterial activity. The sulfonamides (e.g. gantrisin and trimethoprim) are useful for the treatment of Streptococcus pneumoniae, beta-hemolytic streptococci and E. coli, and have been used in the treatment of uncomplicated UTI caused by E. coli, and in the treatment of meningococcal meningitis.

Antiviral agents are compounds which prevent infection of cells by viruses or replication of the virus within the cell. There are many fewer antiviral drugs than antibacterial drugs because the process of viral replication is so closely related to DNA replication within the host cell, that non-specific antiviral agents would often be toxic to the host. There are several stages within the process of viral infection which can be blocked or inhibited by antiviral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleoside analogues), maturation of new virus proteins (e.g. protease inhibitors), and budding and release of the virus.

Nucleotide analogues are synthetic compounds which are similar to nucleotides, but which have an incomplete or abnormal deoxyribose or ribose group. Once the nucleotide analogues are in the cell, they are phosphorylated, producing the triphosphate formed which competes with normal nucleotides for incorporation into the viral DNA or RNA. Once the triphosphate form of the nucleotide analogue is incorporated into the growing nucleic acid chain, it causes irreversible association with the viral polymerase and thus chain termination. Nucleotide analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, and zidovudine (azidothymidine).

The interferons are cytokines which are secreted by virus-infected cells as well as immune cells. The interferons function by binding to specific receptors on cells adjacent to the infected cells, causing the change in the cell which protects it from infection by the virus. α and β-interferon also induce the expression of Class I and Class II MHC molecules on the surface of infected cells, resulting in increased antigen presentation for host immune cell recognition. α and β-interferons are available as recombinant forms and have been used for the treatment of chronic hepatitis B and C infection. At the dosages which are effective for anti-viral therapy, interferons have severe side effects such as fever, malaise and weight loss.

Immunoglobulin therapy is used for the prevention of viral infection. Immunoglobulin therapy for viral infections is different than bacterial infections, because rather than being antigen-specific, the immunoglobulin therapy functions by binding to extracellular virions and preventing them from attaching to and entering cells which are susceptible to the viral infection. The therapy is useful for the prevention of viral infection for the period of time that the antibodies are present in the host. In general there are two types of immunoglobulin therapies, normal immunoglobulin therapy and hyper-immunoglobulin therapy. Normal immune globulin therapy utilizes a antibody product which is prepared from the serum of normal blood donors and pooled. This pooled product contains low titers of antibody to a wide range of human viruses, such as hepatitis A, parvovirus, enterovirus (especially in neonates). Hyper-immune globulin therapy utilizes antibodies which are prepared from the serum of individuals who have high titers of an antibody to a particular virus. Those antibodies are then used against a specific virus. Examples of hyper-immune globulins include zoster immune globulin (useful for the prevention of varicella in immuno-compromised children and neonates), human rabies immunoglobulin (useful in the post-exposure prophylaxis of a subject bitten by a rabid animal), hepatitis B immune globulin (useful in the prevention of hepatitis B virus, especially in a subject exposed to the virus), and RSV immune globulin (useful in the treatment of respiratory syncitial virus infections).

Another type of immunoglobulin therapy is active immunization. This involves the administration of antibodies or antibody fragments to viral surface proteins. Two types of vaccines which are available for active immunization of hepatitis B include serum-derived hepatitis B antibodies and recombinant hepatitis B antibodies. Both are prepared from HBsAg. The antibodies are administered in three doses to subjects at high risk of infection with hepatitis B virus, such as health care workers, sexual partners of chronic carriers, and infants.

Anti-fungal agents are useful for the treatment and prevention of infective fungi. Anti-fungal agents are sometimes classified by their mechanism of action. Some anti-fungal agents function as cell wall inhibitors by inhibiting glucose synthase. These include, but are not limited to, basiungin/ECB. Other anti-fungal agents function by destabilizing membrane integrity. These include, but are not limited to, immidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g. chitinase) or immunosuppression (501 cream). Some examples of commercially-available agents are shown in Table 2.

TABLE 2

| Company | Brand Name | Generic Name | Indication | Mechanism of Action |
|---|---|---|---|---|
| PHARMACIA & | PNU 196443 | PNU 196443 | Anti Fungal | n/k |
| UPJOHN | | | | |
| Lilly | LY 303366 | Basiungin/ECB | Fungal | Anti-fungal/cell wall |
| | | | Infections | inhibitor, glucose |
| | | | | synthase inhibitor |
| Lilly | LY 303366 | Basiungin/ECB | Fungal | Anti-fungal/cell wall |
| | | | Infections | inhibitor, glucose |
| | | | | synthase inhibitor |
| Bayer | Canesten | Clotrimazole | Fungal | Membrane integrity |
| | | | Infections | destabilizer |
| Fujisawa | FK 463 | FK 463 | Fungal | Membrane integrity |
| | | | Infections | destabilizer |
| Mylan | Sertaconzaole | Sertaconzole | Fungal | Membrane integrity |
| | | | Infections | destabilizer |
| Genzyme | Chitinase | Chitinase | Fungal | Chitin Breakdown |
| | | | Infections, | |
| | | | Systemic | |
| Liposome | Abelcet | Amphotericin B, | Fungal | Membrane integrity |
| | | Liposomal | Infections, | destabilizer |
| | | | Systemic | |
| Liposome | Abelcet | Amphotericin B, | Fungal | Membrane integrity |
| | | Liposomal | Infections, | destabilizer |
| | | | Systemic | |
| Sequus | Amphotec | Amphotericin B, | Fungal | Membrane integrity |
| | | Liposomal | Infections, | destabilizer |
| | | | Systemic | |
| Sequus | Amphotec | Amphotericin B, | Fungal | Membrane integrity |
| | | Liposomal | Infections, | destabilizer |
| | | | Systemic | |
| Bayer | BAY 38-9502 | BAY 38-9502 | Fungal | Membrane integrity |
| | | | Infections, | destabilizer |
| | | | Systemic | |
| Pfizer | Diflucan | Fluconazole | Fungal | Membrane integrity |
| | | | Infections, | destabilizer |
| | | | Systemic | |
| Pfizer | Diflucan | Fluconazole | Fungal | Membrane integrity |
| | | | Infections, | destabilizer |
| | | | Systemic | |
| Johnson & | Sporanox | Itraconazole | Fungal | Membrane integrity |
| Johnson | | | Infections, | destabilizer |
| | | | Systemic | |
| Johnson & | Sporanox | Itraconazole | Fungal | Membrane integrity |
| Johnson | | | Infections, | destabilizer |
| | | | Systemic | |
| Sepracor | Itraconzole | Itraconzole | Fungal | Membrane integrity |
| | (2R, 4S) | (2R, 4S) | Infections, | destabilizer |
| | | | Systemic | |
| Johnson & | Nizoral | Ketoconazole | Fungal | Membrane integrity |
| Johnson | | | Infections, | destabilizer |
| | | | Systemic | |
| Johnson & | Nizoral | Ketoconazole | Fungal | Membrane integrity |
| Johnson | | | Infections, | destabilizer |
| | | | Systemic | |
| Johnson & | Monistat | Miconazole | Fungal | Membrane integrity |
| Johnson | | | Infections, | destabilizer |
| | | | Systemic | |
| Johnson & | Monistat | Miconazole | Fungal | Membrane integrity |
| Johnson | | | Infections, | destabilizer |
| | | | Systemic | |
| Merck | MK991 | MK 991 | Fungal | Membrane integrity |
| | | | Infections, | destabilizer |
| | | | Systemic | |
| Merck | MK991 | MK 991 | Fungal | Membrane integrity |
| | | | Infections, | destabilizer |
| | | | Systemic | |
| Bristol | Pradimicin | Pradimicin | Fungal | Membrane integrity |
| Myers Sq'b | | | Infections, | destabilizer |
| | | | Systemic | |
| Pfizer | UK-292, 663 | UK-292, 663 | Fungal | Membrane integrity |
| | | | Infections, | destabilizer |
| | | | Systemic | |
| Pfizer | UK-292, 663 | UK-292, 663 | Fungal | Membrane integrity |
| | | | Infections, | destabilizer |
| | | | Systemic | |
| Pfizer | Voriconazole | Voriconazole | Fungal | Membrane integrity |
| | | | Infections, | destabilizer |
| | | | Systemic | |
| Pfizer | Voriconazole | Voriconazole | Fungal | Membrane integrity |
| | | | Infections, | destabilizer |
| | | | Systemic | |
| Mylan | 501 Cream | 501 Cream | Inflammatory | Immunosuppression |
| | | | Fungal | |
| | | | Conditions | |
| Mylan | Mentax | Butenafine | Nail Fungus | Membrane Integrity |
| | | | | Destabiliser |
| Schering | Anti Fungal | Anti Fungal | Opportunistic | Membrane Integrity |
| Plough | | | Infections | Destabiliser |
| Schering | Anti Fungal | Anti Fungal | Opportunistic | Membrane Integrity |
| Plough | | | Infections | Destabiliser |
| Alza | Mycelex | Clotrimazole | Oral Thrush | Membrane Integrity |
| | Troche | | | Stabliser |
| Novartis | Lamisil | Terbinafine | Systemic | Membrane Integrity |
| | | | Fungal | Destabiliser |
| | | | Infections, | |
| | | | Onychomycosis | |

Diseases associated with fungal infection include aspergillosis, blastomycosis, camdidiais, chromoblastomycosis, coccidioidomycosis, cryptococcosis, fungal eye infections, fungal hair, nail, and skin infections, histoplasmosis, lobomycosis, mycetoma, otomycosis, paracoccidioidomycosis, penicilliosis, marneffeii, phaeohyphomycosis, rhinosporidioisis, sporotrichosis, and zygomycosis.

Aspergillosis is a disease caused by the fungi of the genus aspergillus, which can lead to mild or severe disease, generally depending on factors such as the status of the host immune system. Aspergillus frequently arises as an opportunistic infection in patients having immune-suppressive diseases, or being treated with chemotherapy. Some forms of aspergillus can be treated with prednisone, disodium chromoglycat, nystatin, amphotericin B, itraconazole, or voriconazole.

Blastomycosis is a fungal infection arising from the organism blastomyces dermatitis. The infection initiates in the lungs and usually is disseminated to other body sites, especially the skin and bone. It is treated by amphotericin B, hydroxystilbamidine, itraconazole and voriconazole. When amphotericin B is used, at least 1.5 grams must be given to avoid relapse. However, when the drug is administered with the immunostimulatory nucleic acids of the invention, lower doses can be given without a relapse. Generally hydroxystilbamidine has been used for treating the cutaneous form of the disease but not other forms. When combined with the immunostimulatory nucleic acids of the invention, it can also be used for the treatment of other forms, as well as in lower doses for the cutaneous form.

Candidiasis is a fungal infection caused by a member of the genus candida. The disease can be in the form of allergic, cutaneous, mucocutaneous, or systemic candidiasis. Nystatin is used for the treatment of the cutaneous, mucocutaneous, and allergic diseases. Amphoterizin B is useful for treating this systemic disease. Other drugs useful for the treatment include 5-fluorocytosine, fluconazole, itraconazole and voriconazole.

Chromoblastomycosis is a chronic infection of the skin and subcutaneous tissue. Although the infection is usually localized, parts can disseminate systemically and in particular to the brain. Itraconazole and terbinafine are the drugs used to treat this infection. The principal fungi causing this infection are cladophialophora, carrionii, fonsecaea, compacta, fonsecaea pedrosoi, phialophora, verruceosa, rhinocladiella, aquasbera.

Coccidioidomycosis is a fungal disease of the respiratory tract which can be acute, chronic, severe or fatal. The disease is primarily caused by coccidioides immitis. Amphoterizin B, itraconazole, fluconazole, ketaconazole, and voriconazole are anti-fungal agents that are used for the treatment of this disorder.

Cryptococcosis is a fungal disorder caused by cryptococcus norformans or filobasidiella neoformans. The disease can take the form of a chronic, subacute, acute, pulmonary, systemic, or meningitic disease, following primary infection in the lungs. If the disease spreads from the lungs to the central nervous system, it is usually treated immediately with amphoterizin B and/or 5-fluorocytosine and in some cases fluconazole.

Fungal infections of the eye include mycotit keratitis, and endogenous or extension occulomycosis. Mycotic keratitis is caused by a variety of fungi including acremonium, aspergillus, bipolaris, candida albicans, curvularia, exserohilum, fusarium, and lasiodiplodia. Amphoterizin B is not used for treatment because it irritates the infected tissue. Drugs useful for treating mycotit keratitis include pimaricin and fluconazole. Occulomycosis is generally caused by candida albicans or rhizopus, arrhizus. Amphoterizin B is the anti-fungal agent used for treatment.

Fungal infections of the hair, nail, and skin include onychomycosis, piedra, pityrisis versizolor, tinea barbae, tinea capitis, tinea corporis, tinea cruris, tinea faosa, tinea nigra, tinea unguium. Onychomycosis, which is generally caused by fungi such as acremonium, aspergillus, candida, fusarium, scopulariopisis, onychocola, and scytalidium, can be treated with itraconazole, turbinifine, amphoterizin B, gentian violet, resorcin, iodine, nystatin, thiabendazole, and glutarardehyde. Piedra, which is a colonization of the hair shaft to bifungal organisms such as piedraia and trichosporin, can be treated with keratolytic agents, mild fungicides, fluconazole, and itraconazole. The tineas are various forms of ringworm colonizing different bodily regions. These diseases are generally caused by fungi such as microsporum, trichophyton, and epidermophyton. The tineas can be treated with keratolytic agents, intraconazole, turbinifine, tolnaftate, chlotrimazole, miconazole, econazole, and ketaconzole.

Histoplasmosis (capsulati and duboisii) are fungal infections caused by histoplasma and ajellomyces. Histoplasmosis capsulati can adequately be treated with amphoterizin B, itraconazole or voriconazole. If the subject being treated has AIDS, fluconazole is usually used. Histoplasmosis duboisii once it becomes disseminated, especially to the liver or spleen, is very difficult to treat. Amphoterizin B, itraconazole, fluconazole, and voriconazole are used. When these compounds are combined with the immunostimulatory nucleic acids of the invention, prognosis is improved.

Lobomycosis is a fungal infection caused by lacazia loboi. Lobomycosis is a cutaneous infection which develops into lesions which can be removed by surgery. There are not drugs specifically used for this disorder. Mycetoma is an infection caused by a variety of fungi including eumycotic, acromonium, aspergillus, exophiala, leptos phaeria, madurella, neotestudina, pseudallesheria, and pyrenochieta. The disease involves lesions of the cutaneous and subcutaneous tissues, which can rupture and spread to surrounding tissues. The mycetomas can be treated with ketoconazole, in combination with surgery.

Otomycosis is a fungal ear infection caused by aspergillus or candida. The infection is a superficial infection of the outer ear canal, which is characterized by inflammation, pruritus, scaling, and sever discomfort. It is a chronic recurring mycosis.

Paracoccidioidomycosis is a fungal infection cause by paracoccidioides brasiliensis. The disease originates as a pulmonary infection and can disseminate into the nasal, buccal, and gastrointestinal mucosa. Amphoterizin B and sulfonamides are generally used to treat the disease.

Phaeohyphomycosis is a fungal infection caused by a variety of fungi including cladophialophora, curvularia, bipolaris, exserohilum, exophiala, scedosporium, ochroconis, coniothyrium, phialophora, wangiella, and lasiodiplodia. The infection can be localized or can invade various tissues including the brain, bone, eyes, and skin. Invasion of the brain or bone can be lethal. Generally, phaeohyphomycosis is treated with amphoterizin B and phyfluorocytozine or intaconazole. Rhinosporidiosis is an infection of the mucus membrane caused by rhinosporidium seeberi. Local injection of amphoterizin B is used as treatment.

Sporotrichosis is a chronic infection of the cutaneous tissues, subcutaneous tissues, or lymph system. The infection may also spread to tissues such as bone, muscle, CNS, lungs, and/or genitourinary system. Usually the fungi sporothrix schenckii is inhaled or passed through a lesion in the skin. Sporotrichosis is usually treated with oral potassium iodide, amphoterizin B, or 5-fluorocytozine.

Zygomycosis is a chronic infection caused by conidobolus and basidiobolus ranarum. The disease is treated by potassium iodide and/or amphoterizin B.

Parasiticides are agents that kill parasites directly. Such compounds are known in the art and are generally commercially available. Examples of parasiticides useful for human administration include but are not limited to albendazole, amphotericin B, benznidazole, bithionol, chloroquine HCl, chloroquine phosphate, clindamycin, dehydroemetine, diethylcarbamazine, diloxanide furoate, eflornithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, tinidazole, trimethroprim-sulfamethoxazole, and tryparsamide some of which are used alone or in combination with others.

Parasiticides used in non-human subjects include piperazine, diethylcarbamazine, thiabendazole, fenbendazole, albendazole, oxfendazole, oxibendazole, febantel, levamisole, pyrantel tartrate, pyrantel pamoate, dichlorvos, ivermectin, doramectic, milbemycin oxime, iprinomectin, moxidectin, N-butyl chloride, toluene, hygromycin B thiacetarsemide sodium, melarsomine, praziquantel, epsiprantel, benzimidazoles such as fenbendazole, albendazole, oxfendazole, clorsulon, albendazole, amprolium; decoquinate, lasalocid, monensin sulfadimethoxine; sulfamethazine, sulfaquinoxaline, metronidazole.

Parasiticides used in horses include mebendazole, oxfendazole, febantel, pyrantel, dichlorvos, trichlorfon, ivermectin, piperazine; for S. westeri: ivermectin, benzimiddazoles such as thiabendazole, cambendazole, oxibendazole and fenbendazole. Useful parasiticides in dogs include milbemycin oxine, ivermectin, pyrantel pamoate and the combination of ivermectin and pyrantel. The treatment of parasites in swine can include the use of levamisole, piperazine, pyrantel, thiabendazole, dichlorvos and fenbendazole. In sheep and goats anthelmintic agents include levamisole or ivermectin. Caparsolate has shown some efficacy in the treatment of D. immitis (heartworm) in cats.

Agents used in the prevention and treatment of protozoal diseases in poultry, particularly trichomoniasis, can be administered in the feed or in the drinking water and include protozoacides such as aminonitrothiazole, dimetridazole (Emtryl), nithiazide (Hepzide) and Enheptin. However, some of these drugs are no longer available for use in agrigultural stocks in the USA. Back yard flocks or pigeons not used for food production may be effectively treated with dimetridazole by prescription of a veterinarian (1000 mg/L in drinking water for 5-7 days).

In addition to the use of the immunostimulatory nucleic acids and anti-microbial agents to prevent infection in humans, the methods of the preferred embodiments are particularly well suited for treatment of non-human vertebrates. Non-human vertebrates which exist in close quarters and which are allowed to intermingle as in the case of zoo, farm and research animals are also embraced as subjects for the methods of the invention. Zoo animals such as the felid species including for example lions, tigers, leopards, cheetahs, and cougars; elephants, giraffes, bears, deer, wolves, yaks, non-human primates, seals, dolphins and whales; and research animals such as mice, rats, hamsters and gerbils are all potential subjects for the methods of the invention.

Birds such as hens, chickens, turkeys, ducks, geese, quail, and pheasant are prime targets for many types of infections. Hatching birds are exposed to pathogenic microorganisms shortly after birth. Although these birds are initially protected against pathogens by maternal derived antibodies, this protection is only temporary, and the bird's own immature immune system must begin to protect the bird against the pathogens. It is often desirable to prevent infection in young birds when they are most susceptible. It is also desirable to prevent against infection in older birds, especially when the birds are housed in closed quarters, leading to the rapid spread of disease. Thus, it is desirable to administer the immunostimulatory nucleic acids and anti-microbial agents to birds to prevent infectious disease.

An example of a common infection in chickens is chicken infectious anemia virus (CIAV). CIAV was first isolated in Japan in 1979 during an investigation of a Marek's disease vaccination break (Yuasa et al., 1979, Avian Dis. 23:366-385). Since that time, CIAV has been detected in commercial poultry in all major poultry producing countries (van Bulow et al., 1991, pp. 690-699) in Diseases of Poultry, 9th edition, Iowa State University Press).

CIAV infection results in a clinical disease, characterized by anemia, hemorrhage and immunosuppression, in young susceptible chickens. Atrophy of the thymus and of the bone marrow and consistent lesions of CIAV-infected chickens are also characteristic of CIAV infection. Lymphocyte depletion in the thymus, and occasionally in the bursa of Fabricius, results in immunosuppression and increased susceptibility to secondary viral, bacterial, or fungal infections which then complicate the course of the disease. The immunosuppression may cause aggravated disease after infection with one or more of Marek's disease virus (MDV), infectious bursal disease virus, reticuloendotheliosis virus, adenovirus, or reovirus. It has been reported that pathogenesis of MDV is enhanced by CIAV (DeBoer et al., 1989, p. 28 In Proceedings of the 38th Western Poultry Diseases Conference, Tempe, Ariz.). Further, it has been reported that CIAV aggravates the signs of infectious bursal disease (Rosenberger et al., 1989, Avian Dis. 33:707-713). Chickens develop an age resistance to experimentally induced disease due to CAA. This is essentially complete by the age of 2 weeks, but older birds are still susceptible to infection (Yuasa, N. et al., 1979 supra; Yuasa, N. et al., Arian Diseases 24, 202-209, 1980). However, if chickens are dually infected with CAA and an immunosuppressive agent (IBDV, MDV etc.) age resistance against the disease is delayed (Yuasa, N. et al., 1979 and 1980 supra; Bulow von V. et al., J. Veterinary Medicine 33, 93-116, 1986). Characteristics of CIAV that may potentiate disease transmission include high resistance to environmental inactivation and some common disinfectants. The economic impact of CIAV infection on the poultry industry is clear from the fact that 10% to 30% of infected birds in disease outbreaks die.

Cattle and livestock are also susceptible to infection. Disease which affect these animals can produce severe economic losses, especially amongst cattle. The methods of the invention can be used to protect against infection in livestock, such as cows, horses, pigs, sheep, and goats.

Cows can be infected by bovine viruses. Bovine viral diarrhea virus (BVDV) is a small enveloped positive-stranded RNA virus and is classified, along with hog cholera virus (HOCV) and sheep border disease virus (BDV), in the pestivirus genus. Although, Pestiviruses were previously classified in the Togaviridae family, some studies have suggested their reclassification within the Flaviviridae family along with the flavivirus and hepatitis C virus (HCV) groups (Francki, et al., 1991).

BVDV, which is an important pathogen of cattle can be distinguished, based on cell culture analysis, into cytopathogenic (CP) and noncytopathogenic (NCP) biotypes. The NCP biotype is more widespread although both biotypes can be found in cattle. If a pregnant cow becomes infected with an NCP strain, the cow can give birth to a persistently infected and specifically immunotolerant calf that will spread virus during its lifetime. The persistently infected cattle can succumb to mucosal disease and both biotypes can then be isolated from the animal. Clinical manifestations can include abortion, teratogenesis, and respiratory problems, mucosal disease and mild diarrhea. In addition, severe thrombocytopenia, associated with herd epidemics, that may result in the death of the animal has been described and strains associated with this disease seem more virulent than the classical BVDVs.

Equine herpesviruses (EHV) comprise a group of antigenically distinct biological agents which cause a variety of infections in horses ranging from subclinical to fatal disease. These include Equine herpesvirus-1 (EHV-1), a ubiquitous pathogen in horses. EHV-1 is associated with epidemics of abortion, respiratory tract disease, and central nervous system disorders. Primary infection of upper respiratory tract of young horses results in a febrile illness which lasts for 8 to 10 days. Immunologically experienced mares may be reinfected via the respiratory tract without disease becoming apparent, so that abortion usually occurs without warning. The neurological syndrome is associated with respiratory disease or abortion and can affect animals of either sex at any age, leading to incoordination, weakness and posterior paralysis (Telford, E. A. R. et al., Virology 189, 304-316, 1992). Other EHV's include EHV-2, or equine cytomegalovirus, EHV-3, equine coital exanthema virus, and EHV-4, previously classified as EHV-1 subtype 2.

Sheep and goats can be infected by a variety of dangerous microorganisms including visna-maedi.

Primates such as monkeys, apes and macaques can be infected by simian immunodeficiency virus. Inactivated cell-virus and cell-free whole simian immunodeficiency vaccines have been reported to afford protection in macaques (Stott et al. (1990) Lancet 36:1538-1541; Desrosiers et al. PNAS USA (1989) 86:6353-6357; Murphey-Corb et al. (1989) Science 246:1293-1297; and Carlson et al. (1990) AIDS Res. Human Retroviruses 6:1239-1246). A recombinant HIV gp120 vaccine has been reported to afford protection in chimpanzees (Berman et al. (1990) Nature 345:622-625).

Cats, both domestic and wild, are susceptible to infection with a variety of microorganisms. For instance, feline infectious peritonitis is a disease which occurs in both domestic and wild cats, such as lions, leopards, cheetahs, and jaguars. When it is desirable to prevent infection with this and other types of pathogenic organisms in cats, the methods of the invention can be used to prevent or treat infection in cats.

Domestic cats may become infected with several retroviruses, including but not limited to feline leukemia virus (FeLV), feline sarcoma virus (FeSV), endogenous type C oncornavirus (RD-1 14), and feline syncytia-forming virus (FeSFV). Of these, FeLV is the most significant pathogen, causing diverse symptoms, including lymphoreticular and myeloid neoplasms, anemias, immune mediated disorders, and an immunodeficiency syndrome which is similar to human acquired immune deficiency syndrome (AIDS). Recently, a particular replication-defective FeLV mutant, designated FeLV-AIDS, has been more particularly associated with immunosuppressive properties.

The discovery of feline T-lymphotropic lentivirus (also referred to as feline immunodeficiency) was first reported in Pedersen et al. (1987) Science 235:790-793. Characteristics of FIV have been reported in Yamamoto et al. (1988) Leukemia, December Supplement 2:204S-215S; Yamamoto et al. (1988) Am. J. Vet. Res. 49:1246-1258; and Ackley et al. (1990) J. Virol. 64:5652-5655. Cloning and sequence analysis of FIV have been reported in Olmsted et al. (1989) Proc. Natl. Acad. Sci. USA 86:2448-2452 and 86:4355-4360.

Feline infectious peritonitis (FIP) is a sporadic disease occurring unpredictably in domestic and wild Felidae. While FIP is primarily a disease of domestic cats, it has been diagnosed in lions, mountain lions, leopards, cheetahs, and the jaguar. Smaller wild cats that have been afflicted with FIP include the lynx and caracal, sand cat, and pallas cat. In domestic cats, the disease occurs predominantly in young animals, although cats of all ages are susceptible. A peak incidence occurs between 6 and 12 months of age. A decline in incidence is noted from 5 to 13 years of age, followed by an increased incidence in cats 14 to 15 years old.

Viral, bacterial, and parasitic diseases in fin-fish, shellfish or other aquatic life forms pose a serious problem for the aquaculture industry. Owing to the high density of animals in the hatchery tanks or enclosed marine farming areas, infectious diseases may eradicate a large proportion of the stock in, for example, a fin-fish, shellfish, or other aquatic life forms facility. The fish immune system has many features similar to the mammalian immune system, such as the presence of B cells, T cells, lymphokines, complement, and immunoglobulins. Fish have lymphocyte subclasses with roles that appear similar in many respects to those of the B and T cells of mammals.

Aquaculture species include but are not limited to fin-fish, shellfish, and other aquatic animals. Fin-fish include all vertebrate fish, which may be bony or cartilaginous fish, such as, for example, salmonids, carp, catfish, yellowtail, seabream, and seabass. Salmonids are a family of fin-fish which include trout (including rainbow trout), salmon, and Arctic char. Examples of shellfish include, but are not limited to, clams, lobster, shrimp, crab, and oysters. Other cultured aquatic animals include, but are not limited to eels, squid, and octopi.

In some cases it is desirable to administer an antigen with the immunostimulatory nucleic acid and the anti-microbial agent and in other cases no antigen is delivered. The antigen, if used, is preferably a microbial antigen. Microbial antigens include, but are not limited to, cells, cell extracts, proteins, polypeptides, peptides, polysaccharides, polysaccharide conjugates, peptide and non-peptide mimics of polysaccharides and other molecules, small molecules, lipids, glycolipids, and carbohydrates. Many microbial antigens, however, are protein or polypeptide in nature, as proteins and polypeptides are generally more antigenic than carbohydrates or fats. Methods for administering an antigen to a subject are well-known in the art. In general, an antigen is administered directly to the subject by any means, such as, e.g., intravenous, intramuscular, oral, transdermal, mucosal, intranasal, intratracheal, or subcutaneous administration. The antigen can be administered systemically or locally. In some preferred embodiments, the antigen is not conjugated to the immunostimulatory nucleic acid. Administration methods are described in more detail below.

The term “substantially purified” as used herein refers to a molecular species which is substantially free of other proteins, lipids, carbohydrates or other materials with which it is naturally associated. One skilled in the art can purify polypeptides, e.g. antigens, using standard techniques for protein purification. The substantially pure polypeptide will often yield a single major band on a non-reducing polyacrylamide gel. In the case of partially glycosylated polypeptides or those that have several start codons, there may be several bands on a non-reducing polyacrylamide gel, but these will form a distinctive pattern for that polypeptide. The purity of the polypeptide can also be determined by amino-terminal amino acid sequence analysis.

The microbial antigen, if administered and if it is a polypeptide, may be in the form of a polypeptide when administered to the subject or it may be encoded by a nucleic acid vector. If the nucleic acid vector is administered to the subject the protein is expressed in vivo. Minor modifications of the primary amino acid sequences of polypeptide microbial antigens may also result in a polypeptide which has substantially equivalent antigenic activity, as compared to the unmodified counterpart polypeptide. Such modifications may be deliberate, as by site-directed mutagenesis, or may be spontaneous. Thus, nucleic acids having such modifications are also encompassed. When an antigen that is encoded by a nucleic acid vector is administered, the immunostimulatory nucleic acid is not the same plasmid or expression vector containing the antigen.

The nucleic acid encoding the antigen is operatively linked to a gene expression sequence which directs the expression of the protein within a eukaryotic cell. The “gene expression sequence” is any regulatory nucleotide sequence, such as a promoter sequence or promoter-enhancer combination, which facilitates the efficient transcription and translation of the protein which it is operatively linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPTR), adenosine deaminase, pyruvate kinase, b-actin promoter and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In general, the gene expression sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined antigen nucleic acid. The gene expression sequences optionally include enhancer sequences or upstream activator sequences as desired.

As used herein, the nucleic acid sequence encoding the protein and the gene expression sequence are said to be “operably linked” when they are covalently linked in such a way as to place the expression or transcription and/or translation of the antigen coding sequence under the influence or control of the gene expression sequence. Two DNA sequences are said to be operably linked if induction of a promoter in the 5′ gene expression sequence results in the transcription of the gene sequence and if the nature of the linkage between the two DNA sequences does not (1) result in the introduction of a frame-shift mutation, (2) interfere with the ability of the promoter region to direct the transcription of the antigen sequence, or (3) interfere with the ability of the corresponding RNA transcript to be translated into a protein. Thus, a gene expression sequence would be operably linked to a specific nucleic acid sequence if the gene expression sequence were capable of effecting transcription of that nucleic acid sequence such that the resulting transcript is translated into the desired protein or polypeptide.

Drug resistance is developing into a major problem in the control and treatment of infectious disease. Since the first antibiotic, penicillin, was introduced in the early 1900s, many strains of clinically-important bacteria, including staphylococci, enterococci, pseudomonas, and pneumococci have become resistant to many antibiotics. It has been reported by the CDC that these classes of bacteria are responsible for almost half of all hospital-acquired infections. Thus, it is important to prevent the development of further antibiotic resistant strains. The increasing incidence of antibiotic resistant strains of bacteria results from the over-use and misuse of antibiotics. When the bacteria or fungi are exposed to the anti-microbial agent, all of the susceptible microbes will be killed, but any that have undergone a genetic change which confers drug resistance, will obtain a selective growth advantage. These microbes will thrive and develop into a new strain. Some of the factors contributing to the misuse of anti-microbial agents that lead to resistant strains include the use of antibacterial drugs to treat non-bacterial infections, the prophylactic use of anti-microbial agents alone to prevent potential but unconfirmed infections, the use of anti-microbial drugs, which have broad spectrum to treat an infection before the disease-causing organism has been identified, misuse by the patient by early termination or other inappropriate use of the anti-microbial agent and long-term anti-microbial therapy for patients who are immunosuppressed and unable on their own to clear infections, such as patients having organ transplants or cancer chemotherapy or diseases such as AIDS.

There are several bases for bacterial and fungal resistance to anti-microbial agents. These include inherent resistance, acquired resistance, vertical evolution, and horizontal evolution. Microorganisms can be inherently resistant to an anti-microbial agent because it has some permeability barrier or other mechanism which prevents it from being effected by the anti-microbial agent. When a microorganism is inherently resistant to an anti-microbial agent, the anti-microbial agent is said to be non-effective for the treatment of that microorganism. A microorganism which acquires resistance is one which develops from some sort of genetic alteration which prevents the microorganism from responding, even though the majority of microorganisms of that strain are sensitive to a particular anti-microbial agent. The genetic change or alteration can arise from mutation and selection, which is referred to as vertical evolution or by exchange of genes between strains and species, which is referred to as horizontal evolution.

The major problem associated with anti-microbial drug resistance is that the particular anti-microbial agent is then useless in the treatment of the infection by the microorganism. As this resistance develops, additional therapies need to be identified or the infection, which was once manageable will become serious and untreatable.

The immunostimulatory nucleic acids of the invention are useful for the prevention of anti-microbial resistance. When the immunostimulatory nucleic acids are administered in conjunction with the anti-microbial agent, surprisingly, it was found that resistant strains were prevented from developing. The term “in conjunction with” as used with respect to this aspect of the invention refers to the administration of the immunostimulatory nucleic acids before, at the same time as, or after the anti-microbial agent as long as it is within a time period that is sufficient to prevent the drug resistance. Preferably, the immunostimulatory nucleic acid is administered within two days more preferably, within one day or within six hours of the anti-microbial agent. Although applicants are not bound by the mechanism, it is believed that the ability of the immunostimulatory nucleic acids to prevent the development of resistant strains results from the ability of the nucleic acids to induce an immune response leading to an improved response by the immune system against a microorganism. At the same time, the anti-microbial agent is functioning to kill or inhibit the microorganism. This dual action may result in rapid inhibition of the invading microorganism, reducing the time in which genetic modifications can occur prior to cell death or inhibition.

The effective amount for preventing drug resistant strains from developing of the immunostimulatory nucleic acid is that amount which is capable of preventing altogether the development of drug resistant strains, inhibiting an increase in the number of drug resistant strains developing, or causing the development of fewer drug resistant strains than would otherwise develop in the absence of the immunostimulatory nucleic acids.

In yet another aspect of the invention, the immunostimulatory nucleic acids are administered to a subject in order to inhibit or prevent an allergic reaction in the subject to an anti-microbial agent. The immunostimulatory nucleic acid is administered in an amount effective to prevent the allergic reaction to the anti-microbial agent. Allergic reactions to many types of anti-microbial agents (the most common probably being penicillin) is a major obstacle to the use of such anti-microbials. Surprisingly, administration of immunostimulatory nucleic acids, particularly those that shift the immune response to a Th1 response from a Th2 response, are particularly effective at reducing the allergic response to such anti-microbials.

The compositions of the invention may be delivered to the immune system or other target cells alone or in association with a vector. In its broadest sense, a “vector” is any vehicle capable of facilitating the transfer of the compositions to the target cells. The vector generally transports the nucleic acid and/or anti-microbial agent to the target cells with reduced degradation relative to the extent of degradation that would result in the absence of the vector. When delivered via such a vector, it is not required that the nucleic acid and the anti-microbial agent be conjugated to each other.

In general, the vectors useful in the invention are divided into two classes: biological vectors and chemical/physical vectors. Biological vectors and chemical/physical vectors are useful for delivery/uptake of nucleic acids, anti-microbial agents, and/or allergens to/by a target cell.

Biological vectors include, but are not limited to, plasmids, phagemids, viruses, other vehicles derived from viral or bacterial sources that have been manipulated by the insertion or incorporation of nucleic acid sequences, and free nucleic acid fragments which can be attached to nucleic acid sequences. Viral vectors are a preferred type of biological vector and include, but are not limited to, nucleic acid sequences from the following viruses: retroviruses, such as: Moloney murine leukemia virus; Harvey murine sarcoma virus; murine mammary tumor virus; Rous sarcoma virus; adenovirus; adeno-associated virus; SV40-type viruses; polyoma viruses; Epstein-Barr viruses; papilloma viruses; herpes viruses; vaccinia viruses; polio viruses; and RNA viruses such as any retrovirus. One can readily employ other viral vectors not named but known in the art.

Preferred viral vectors are based on non-cytopathic eukaryotic viruses in which non-essential genes have been replaced with a nucleic acid of interest. Non-cytopathic viruses include retroviruses, the life cycle of which involves reverse transcription of genomic viral RNA into DNA with subsequent proviral integration into host cellular DNA. Retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman Co., New York (1990) and Murry, E. J. Ed. “Methods in Molecular Biology,” vol. 7, Humana Press, Inc., Cliffton, N.J. (1991).

Another preferred virus for certain applications is the adeno-associated virus, a double-stranded DNA virus. The adeno-associated virus can be engineered to be replication-deficient and is capable of infecting a wide range of cell types and species. It further has advantages, such as heat and lipid solvent stability; high transduction frequencies in cells of diverse lineages; and lack of superinfection inhibition thus allowing multiple series of transductions. Reportedly, the adeno-associated virus can integrate into human insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other biological vectors include plasmid vectors. Plasmid vectors have been extensively described in the art and are well-known to those of skill in the art. See e.g., Sambrook et al., “Molecular Cloning: A Laboratory Manual,” Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well-known to those of ordinary skill in the art. Additionally, plasmids may be custom designed using restriction enzymes and ligation reactions to remove and add specific fragments of DNA.

It has recently been discovered that gene carrying plasmids can be delivered to the immune system using bacteria. Modified forms of bacteria such as Salmonella can be transfected with the plasmid and used as delivery vehicles. The bacterial delivery vehicles can be administered to a host subject orally or by other administration means. The bacteria deliver the plasmid to immune cells, e.g. B cells, dendritic cells, likely by passing through the gut barrier. High levels of immune protection have been established using this methodology. Such methods of delivery are useful for the aspects of the invention utilizing systemic delivery of immunostimulatory nucleic acid, anti-microbial agent and/or other therapeutic agent.

In addition to the biological vectors, chemical/physical vectors may be used to deliver a nucleic acid, anti-microbial agent to a target cell and facilitate uptake thereby. As used herein, a “chemical/physical vector” refers to a natural or synthetic molecule, other than those derived from bacteriological or viral sources, capable of delivering the nucleic acid and/or anti-microbial agent to a cell.

A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vessels (LUV), which range in size from 0.2-4.0 μm can encapsulate large macromolecules. RNA, DNA, and intact virions can be encapsulated within the aqueous interior and be delivered to cells in a biologically active form (Fraley, et al., Trends Biochem. Sci., (1981) 6:77).

Liposomes may be targeted to a particular tissue by coupling the liposome to a specific ligand such as a monoclonal antibody, sugar, glycolipid, or protein. Ligands which may be useful for targeting a liposome to an immune cell include, but are not limited to: intact or fragments of molecules which interact with immune cell specific receptors and molecules, such as antibodies, which interact with the cell surface markers of immune cells. Such ligands may easily be identified by binding assays well known to those of skill in the art. Additionally, the vector may be coupled to a nuclear targeting peptide, which will direct the vector to the nucleus of the host cell.

Lipid formulations for transfection are commercially available from QIAGEN, for example, as EFFECTENE™ (a non-liposomal lipid with a special DNA condensing enhancer) and SUPERFECT™ (a novel acting dendrimeric technology).

Liposomes are commercially available from Gibco BRL, for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes also have been reviewed by Gregoriadis, G. in Trends in Biotechnology, (1985) 3:235-241.

In one embodiment, the vehicle is a biocompatible microparticle or implant that is suitable for implantation or administration to the mammalian recipient. Exemplary bioerodible implants that are useful in accordance with this method are described in PCT International application no. PCT/US/03307 (Publication No. WO95/24929, entitled “Polymeric Gene Delivery System”. PCT/US/0307 describes a biocompatible, preferably biodegradable polymeric matrix for containing an exogenous gene under the control of an appropriate promoter. The polymeric matrix can be used to achieve sustained release of the exogenous gene in the patient.

The polymeric matrix preferably is in the form of a microparticle such as a microsphere (wherein the a nucleic acid, anti-microbial agent, and/or allergen is dispersed throughout a solid polymeric matrix) or a microcapsule (wherein the a nucleic acid, anti-microbial agent, and/or allergen is stored in the core of a polymeric shell). Other forms of the polymeric matrix for containing the a nucleic acid, anti-microbial agent, and/or allergen include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, typically injection into a tissue or administration of a suspension by aerosol into the nasal and/or pulmonary areas. Preferably when an aerosol route is used the polymeric matrix and the nucleic acid, anti-microbial agent, and/or allergen are encompassed in a surfactant vehicle. The polymeric matrix composition can be selected to have both favorable degradation rates and also to be formed of a material which is bioadhesive, to further increase the effectiveness of transfer when the matrix is administered to a nasal and/or pulmonary surface that has sustained an injury. The matrix composition also can be selected not to degrade, but rather, to release by diffusion over an extended period of time.

In another embodiment the chemical/physical vector is a biocompatible microsphere that is suitable for delivery, such as oral or mucosal delivery. Such microspheres are disclosed in Chickering et al., Biotech. And Bioeng., (1996) 52:96-101 and Mathiowitz et al., Nature, (1997) 386:.410-414 and PCT Patent Application WO97/03702.

Both non-biodegradable and biodegradable polymeric matrices can be used to deliver the nucleic acid and/or anti-microbial to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

Bioadhesive polymers of particular interest include bioerodible hydrogels described by H. S. Sawhney, C. P. Pathak and J. A. Hubell in Macromolecules, (1993) 26:581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Compaction agents also can be used alone, or in combination with, a biological or chemical/physical vector to deliver nucleic acids. A “compaction agent”, as used herein, refers to an agent, such as a histone, that neutralizes the negative charges on the nucleic acid and thereby permits compaction of the nucleic acid into a fine granule. Compaction of the nucleic acid facilitates the uptake of the nucleic acid by the target cell. The compaction agents can be used alone, i.e., to deliver a nucleic acid in a form that is more efficiently taken up by the cell or, more preferably, in combination with one or more of the above-described vectors.

Other exemplary compositions that can be used to facilitate uptake by a target cell of the nucleic acid and/or anti-microbial include calcium phosphate and other chemical mediators of intracellular transport, microinjection compositions, electroporation and homologous recombination compositions (e.g., for integrating a nucleic acid into a preselected location within the target cell chromosome).

The immunostimulatory nucleic acid and/or the anti-microbial and/or other therapeutics may be administered alone (e.g. in saline or buffer) or using any delivery vectors known in the art. For instance the following delivery vehicles have been described: Cochleates (Gould-Fogerite et al., 1994, 1996); Emulsomes (Vancott et al., 1998, Lowell et al., 1997); ISCOMs (Mowat et al., 1993, Carlsson et al., 1991, Hu et., 1998, Morein et al., 1999); Liposomes (Childers et al., 1999, Michalek et al., 1989, 1992, de Haan 1995a, 1995b); Live bacterial vectors (e.g., Salmonella, Escherichia coli, Bacillus calmatte-guerin, Shigella, Lactobacillus) (Hone et al., 1996, Pouwels et al., 1998, Chatfield et al., 1993, Stover et al., 1991, Nugent et al., 1998); Live viral vectors (e.g., Vaccinia, adenovirus, Herpes Simplex) (Gallichan et al., 1993, 1995, Moss et al., 1996, Nugent et al., 1998, Flexner et al., 1988, Morrow et al., 1999); Microspheres (Gupta et al., 1998, Jones et al., 1996, Maloy et al., 1994, Moore et al., 1995, O'Hagan et al., 1994, Eldridge et al., 1989); Nucleic acid vaccines (Fynan et al., 1993, Kuklin et al., 1997, Sasaki et al., 1998, Okada et al., 1997, Ishii et al., 1997); Polymers (e.g. carboxymethylcellulose, chitosan) (Hamajima et al., 1998, Jabbal-Gill et al., 1998); Polymer rings (Wyatt et al., 1998); Proteosomes (Vancott et al., 1998, Lowell et al., 1988, 1996, 1997); Sodium Fluoride (Hashi et al., 1998); Transgenic plants (Tacket et al., 1998, Mason et al., 1998, Haq et al., 1995); Virosomes (Gluck et al., 1992, Mengiardi et al., 1995, Cryz et al., 1998); Virus-like particles (Jiang et al., 1999, Leibl et al., 1998).

The immunostimulatory nucleic acid and anti-microbial agent can be combined with other therapeutic agents such as adjuvants to enhance immune responses even further. The immunostimulatory nucleic acid, and/or anti-microbial agent and/or other therapeutic agent may be administered simultaneously or sequentially. When the other therapeutic agents are administered simultaneously they can be administered in the same or separate formulations, but are administered at the same time. The other therapeutic agents are administered sequentially with one another and with the immunostimulatory nucleic acid anti-microbial agent, when the administration of the other therapeutic agents and the immunostimulatory nucleic acid and anti-microbial agent is temporally separated. The separation in time between the administration of these compounds may be a matter of minutes or it may be longer. Other therapeutic agents include but are not limited to non-nucleic acid adjuvants, cytokines, antibodies, antigens, etc. Preferably, treatment with an anti-viral agent is precluded if a CpG immunostimulatory nucleic acid is used in conjunction with an adjuvant.

A “non-nucleic acid adjuvant” is any molecule or compound except for the immunostimulatory nucleic acids described herein which can stimulate the humoral and/or cellular immune response. Non-nucleic acid adjuvants include, for instance, adjuvants that create a depo effect, immune stimulating adjuvants, adjuvants that create a depo effect and stimulate the immune system and mucosal adjuvants.

An “adjuvant that creates a depo effect” as used herein is an adjuvant that causes an antigen to be slowly released in the body, thus prolonging the exposure of immune cells to the antigen. This class of adjuvants includes but is not limited to alum (e.g., aluminum hydroxide, aluminum phosphate); or emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720, AirLiquide, Paris, France); MF-59 (a squalene-in-water emulsion stabilized with Span 85 and Tween 80; Chiron Corporation, Emeryville, Calif.; and PROVAX (an oil-in-water emulsion containing a stabilizing detergent and a micelle-forming agent; IDEC, Pharmaceuticals Corporation, San Diego, Calif.).

An “immune stimulating adjuvant” is an adjuvant that causes activation of a cell of the immune system. It may, for instance, cause an immune cell to produce and secrete cytokines. This class of adjuvants includes but is not limited to saponins purified from the bark of the Q. saponaria tree, such as QS21 (a glycolipid that elutes in the 21^{st} peak with HPLC fractionation; Aquila Biopharmaceuticals, Inc., Worcester, Mass.); poly[di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.).

“Adjuvants that create a depo effect and stimulate the immune system” are those compounds which have both of the above-identified functions. This class of adjuvants includes but is not limited to ISCOMS (Immunostimulating complexes which contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21: SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxpropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

A “non-nucleic acid mucosal adjuvant” as used herein is an adjuvant other than an immunostimulatory nucleic acid that is capable of inducing a mucosal immune response in a subject when administered to a mucosal surface in conjunction with an antigen. Mucosal adjuvants include but are not limited to Bacterial toxins: e.g., Cholera toxin (CT), CT derivatives including but not limited to CT B subunit (CTB) (Wu et al., 1998, Tochikubo et al., 1998); CTD53 (Val to Asp) (Fontana et al., 1995); CTK97 (Val to Lys) (Fontana et al., 1995); CTK104 (Tyr to Lys) (Fontana et al., 1995); CTD53/K63 (Val to Asp, Ser to Lys) (Fontana et al., 1995); CTH54 (Arg to His) (Fontana et al., 1995); CTN107 (His to Asn) (Fontana et al., 1995); CTE114 (Ser to Glu) (Fontana et al., 1995); CTE112K (Glu to Lys) (Yamamoto et al., 1997a); CTS61F (Ser to Phe) (Yamamoto et al., 1997a, 1997b); CTS106 (Pro to Lys) (Douce et al., 1997, Fontana et al., 1995); andCTK63 (Ser to Lys) (Douce et al., 1997, Fontana et al., 1995), Zonula occludens toxin, zot, Escherichia coli heat-labile enterotoxin, Labile Toxin (LT), LT derivatives including but not limited to LTB subunit (LTB) (Verweij et al., 1998); LT7K (Arg to Lys) (Komase et al., 1998, Douce et al., 1995); LT61F (Ser to Phe) (Komase et al., 1998); LT112K (Glu to Lys) (Komase et al., 1998); LT 118E (Gly to Glu) (Komase et al., 1998); LT146E (Arg to Glu) (Komase et al., 1998); LT192G (Arg to Gly) (Komase et al., 1998); LTK63 (Ser to Lys) (Marchetti et al., 1998, Douce et al., 1997, 1998, Di Tommaso et al., 1996); and LTR72 (Ala to Arg) (Giuliani et al., 1998), Pertussis toxin, PT. (Lycke et al., 1992, Spangler BD, 1992, Freytag and Clemments, 1999, Roberts et al., 1995, Wilson et al., 1995) including PT-9K/129G (Roberts et al., 1995, Cropley et al., 1995); Toxin derivatives (see below) (Holmgren et al., 1993, Verweij et al., 1998, Rappuoli et al., 1995, Freytag and Clements, 1999); Lipid A derivatives (e.g., monophosphoryl lipid A, MPL) (Sasaki et al., 1998, Vancott et al., 1998; Muramyl Dipeptide (MDP) derivatives (Fukushima et al., 1996, Ogawa et al., 1989, Michalek et al., 1983, Morisaki et al., 1983); Bacterial outer membrane proteins (e.g., outer surface protein A (OspA) lipoprotein of Borrelia burgdorferi, outer membrane protine of Neisseria meningitidis)(Marinaro et al., 1999, Van de Verg et al., 1996); Oil-in-water emulsions (e.g., MF59) (Barchfield et al., 1999, Verschoor et al., 1999, O'Hagan, 1998); Aluminum salts (Isaka et al., 1998, 1999); and Saponins (e.g., QS21) Aquila Biopharmaceuticals, Inc., Worcester, Mass.) (Sasaki et al., 1998, MacNeal et al., 1998), ISCOMS, MF-59 (a squalene-in-water emulsion stabilized with Span 85 and Tween 80; Chiron Corporation, Emeryville, Calif.); the Seppic ISA series of Montanide adjuvants (e.g., Montanide ISA 720; AirLiquide, Paris, France); PROVAX (an oil-in-water emulsion containing a stabilizing detergent and a micell-forming agent; IDEC Pharmaceuticals Corporation, San Diego, Calif.); Syntext Adjuvant Formulation (SAF; Syntex Chemicals, Inc., Boulder, Colo.); poly[di(carboxylatophenoxy)phosphazene (PCPP polymer; Virus Research Institute, USA) and Leishmania elongation factor (Corixa Corporation, Seattle, Wash.).

Immune responses can also be induced or augmented by the co-administration or co-linear expression of cytokines (Bueler & Mulligan, 1996; Chow et al., 1997; Geissler et al., 1997; Iwasaki et al., 1997; Kim et al., 1997) or B-7 co-stimulatory molecules (Iwasaki et al., 1997; Tsuji et al., 1997) with the immunostimulatory nucleic acids and anti-microbial agents. The cytokines can be administered directly with immunostimulatory nucleic acids or may be administered in the form of a nucleic acid vector that encodes the cytokine, such that the cytokine can be expressed in vivo. In one embodiment, the cytokine is administered in the form of a plasmid expression vector. In this embodiment, the immunostimulatory nucleic acid is not contained within the same plasmid. The term “cytokine” is used as a generic name for a diverse group of soluble proteins and peptides which act as humoral regulators at nano-to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to IL-1, IL-2, IL-4, IL-5, IL-6, IL-7, IL-10, IL-12, IL-15, IL-18 granulocyte-macrophage colony stimulating factor (GM-CSF), granulocyte colony stimulating factor (GCSF), interferon-γ (γ-IFN), IFN-a, tumor necrosis factor (TNF), TGF-β, FLT-3 ligand, and CD40 ligand. Cytokines play a role in directing the T cell response. Helper (CD4+) T cells orchestrate the immune response of mammals through production of soluble factors that act on other immune system cells, including other T cells. Most mature CD4+ T helper cells express one of two cytokine profiles: Th1 or Th2. In some embodiments it is preferred that the cytokine be a Th1 cytokine.

The term “effective amount” of an immunostimulatory nucleic acid and an anti-microbial agent refers to the amount necessary or sufficient to realize a desired biologic effect. For example, an effective amount of an immunostimulatory nucleic acid and an anti-microbial agent for treating or preventing infectious disease is that amount necessary to prevent the infection with the microorganism if the subject is not yet infected or is that amount necessary to prevent an increase in infected cells or microorganisms present in the subject or that amount necessary to decrease the amount of the infection that would otherwise occur in the absence of the immunostimulatory nucleic acid or anti-microbial agent when either is used alone. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular immunostimulatory nucleic acid or anti-microbial agent being administered (e.g. the type of nucleic acid, i.e. a CpG nucleic acid, the number of immunostimulatory motifs or their location in the nucleic acid, the degree of modification of the backbone to the oligonucleotide the type of medicament), the size of the subject, or the severity of the disease or condition. One of ordinary skill in the art can empirically determine the effective amount of a particular immunostimulatory nucleic acid and/or anti-microbial agent and/or other therapeutic agent without necessitating undue experimentation.

In some embodiments of the invention, the immunostimulatory nucleic acid and anti-microbial agent are administered in a synergistic amount effective to treat or prevent infectious disease. A synergistic amount is that amount which produces a physiological response that is greater than the sum of the individual effects of either the immunostimulatory nucleic acid or the anti-microbial agent alone. For instance, in some embodiments of the invention, the physiological effect is a reduction in the number of cells infected with the virus. A synergistic amount is that amount which produces a reduction in infected cells that is greater than the sum of the infected cells reduced by either the immunostimulatory nucleic acid or the anti-microbial agent alone. In other embodiments, the physiological result is a reduction in the number of microorganisms in the body. The synergistic amount in this case is that amount which produces the reduction that is greater than the sum of the reduction produced by either the immunostimulatory nucleic acid or the anti-microbial agent alone. In other embodiments the physiological result is a decrease in physiological parameters associated with the infection, e.g., fungal lesions or other symptoms. For instance, a diagnosis of urinary tract infection is based on the presence and quantification of bacteria in the urine when greater than 10⁵ colonies per milliliter of microorganisms are detected in a mid-stream, clean-voided urine specimen. A reduction in this number to 10³ and preferably to fewer than 10² bacterial colonies per milliliter indicates that the infection has been eradicated.

Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 0.1 μg to 20 mg/kg/day, more typically from about 1 to 10 mg/kg/day, and most typically from about 1 to 5 mg/kg/day.

In some instances, a sub-therapeutic dosage of the immunostimulatory nucleic acid and the anti-microbial agent are used. It has been discovered according to the invention, that when the two classes of drugs are used together, they can be administered in sub-therapeutic doses and still produce a desirable therapeutic result, a “sub-therapeutic dose” as used herein refers to a dosage which is less than that dosage which would produce a therapeutic result in the subject. Thus, the sub-therapeutic dose of an anti-microbial agent is one which would not produce the desired therapeutic result in the subject in the absence of the immunostimulatory nucleic acid. Therapeutic doses of anti-microbial agent are well known in the field of medicine for the treatment of infectious disease. These dosages have been extensively described in references such as Remington's Pharmaceutical Sciences, 18th ed., 1990; as well as many other medical references relied upon by the medical profession as guidance for the treatment of infectious disease. Therapeutic dosages of immunostimulatory nucleic acids, have also been described in the art and methods for identifying therapeutic dosages in subjects are described in more detail above.

In other aspects, the method of the invention involves administering a high dose of an anti-microbial agent to a subject, without inducing side effects. Ordinarily, when an anti-microbial agent is administered in a high dose, a variety of side effects can occur. (Discussed in more detail above, as well as in the medical literature). As a result of these side effects, the anti-microbial agent is not administered in such high doses, no matter what therapeutic benefits are derived. It was discovered, according to the invention, that such high doses of anti-microbial agents which ordinarily induce side effects can be administered without inducing the side effects as long as the subject also receives an immunostimulatory nucleic acid. The type and extent of the side effects ordinarily induced by the anti-microbial agent will depend on the particular anti-microbial agent used.

In other embodiments of the invention, the immunostimulatory nucleic acid is administered on a routine schedule. The anti-microbial agent may also be administered on a routine schedule, but alternatively, may be administered as symptoms arise. A “routine schedule” as used herein, refers to a predetermined designated period of time. The routine schedule may encompass periods of time which are identical or which differ in length, as long as the schedule is predetermined. For instance, the routine schedule may involve administration of the immunostimulatory nucleic acid on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve administration of the immunostimulatory nucleic acid on a daily basis for the first week, followed by a monthly basis for several months, and then every three months after that. Any particular combination would be covered by the routine schedule as long as it is determined ahead of time that the appropriate schedule involves administration on a certain day.

In other aspects, the invention relates to kits that are useful in the treatment of infectious disease. One kit of the invention includes a container housing an immunostimulatory nucleic acid and a container housing an anti-microbial agent and instructions for timing of administration of the immunostimulatory nucleic acid and the anti-microbial agent. Preferably, the immunostimulatory nucleic acid is provided for systemic administration, and the instructions accordingly provide for this. In an important embodiment, the container housing the immunostimulatory nucleic acid is a sustained release vehicle is used herein in accordance with its prior art meaning of any device which slowly releases the immunostimulatory nucleic acid.

Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation. Another suitable compound for sustained release delivery is GELFOAM, a commercially available product consisting of modified collagen fibers.

The anti-microbial agent is housed in at least one container. The container may be a single container housing all of the anti-microbial agent together or it may be multiple containers or chambers housing individual dosages of the anti-microbial agent, such as a blister pack. The kit also has instructions for timing of administration of the anti-microbial agent. The instructions would direct the subject having an infectious disease or at risk of an infectious disease to take the anti-microbial agent at the appropriate time. For instance, the appropriate time for delivery of the medicament may be as the symptoms occur. Alternatively, the appropriate time for administration of the medicament may be on a routine schedule such as monthly or yearly.

Another kit of the invention includes at least one container housing an immunostimulatory nucleic acid and at least one container housing an anti-microbial agent and instructions for administering the compositions in effective amounts for inducing a synergistic immune response in the subject. The immunostimulatory nucleic acid and anti-microbial agent may be housed in single containers or in separate compartments or containers, such as single dose compartments. The instructions in the kit direct the subject to take the immunostimulatory nucleic acid and the anti-microbial agent in amounts which will produce a synergistic immune response. The drugs may be administered simultaneously or separately as long as they are administered close enough in time to produce a synergistic response.

In other aspects of the invention, a composition is provided. The composition includes an immunostimulatory nucleic and an anti-microbial agent formulated in a pharmaceutically-acceptable carrier and present in the composition in an effective amount for preventing or treating an infectious disease. The effective amount for preventing or treating an infectious disease is that amount which prevents, inhibits completely or partially infection or prevents an increase in the infection. In another aspect, the composition provides an immunostimulatory nucleic acid in an effective amount to prevent or inhibit an allergic reaction to an anti-microbial agent, which may also be present in the composition. Alternatively, the immunostimulatory nucleic acid and the anti-microbial agent may be present (in the same respective doses for preventing or inhibiting an allergic response) separately in a kit.

For any compound described herein a therapeutically effective amount can be initially determined from cell culture assays and based on known effective amounts for known nucleic acids and anti-microbial agents. For instance the effective amount of immunostimulatory nucleic acid useful for inducing B cell activation can be assessed using the in vitro assays with respect to stimulation index in comparison to known immunostimulatory acids. The stimulation index can be used to determine an effective amount of the particular oligonucleotide for the particular subject, and the dosage can be adjusted upwards or downwards to achieve the desired levels in the subject.

Therapeutically effective amounts can also be determined from animal models. A therapeutically effective dose can also be determined from human data for immunostimulatory nucleic acids which have been tested in humans and for compounds which are known to exhibit similar pharmacological activities, such as other adjuvants, e.g., LT for vaccination purposes. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan. Most of the anti-microbial agents have been identified. These amounts can be adjusted when they are combined with immuno-stimulatory nucleic acids by routine experimentation, based on the teachings within the specification.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

Anti-microbial agents and immunostimulatory nucleic acids can be administered by any ordinary route for administering medications. Preferably, they are inhaled, ingested or administered by systemic routes. Systemic routes include oral and parenteral. Inhaled medications are preferred in some embodiments because of the direct delivery to the lung, e.g. when bacterial, viral or fungal agents are inhaled. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers.

For use in therapy, an effective amount of the immunostimulatory nucleic acid can be administered to a subject by any mode that delivers the nucleic acid to the desired surface, e.g., mucosal, systemic. “Administering” the pharmaceutical composition of the present invention may be accomplished by any means known to the skilled artisan. Preferred routes of administration include but are not limited to oral, parenteral, intramuscular, intranasal, intratracheal, inhalation, ocular, vaginal, and rectal.

For oral administration, the compounds (i.e., immunostimulatory nucleic acids, anti-microbial agent, other therapeutic agent) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the therapeutic, such as the immunostimulatory capacity of the nucleic acids (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The immunostimulatory nucleic acids and anti-microbial agent may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of an immunostimulatory nucleic acid and optionally anti-microbial agent and/or other therapeutic agents optionally included in a pharmaceutically-acceptable carrier. The term “pharmaceutically-acceptable carrier” means one or more compatible solid or liquid filler, dilutants or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

We claim:

1. A method for treating or preventing an infectious disease in a subject having or at risk of developing the infectious disease, comprising
administering to a subject in need of such treatment a poly-G nucleic acid and an anti-microbial agent in an effective amount for treating or preventing the infectious disease,
wherein the poly-G nucleic acid is not conjugated to the anti-microbial agent.

2. The method of claim 1, wherein the effective amount is a synergistic amount.

3. The method of claim 1, wherein the poly-G nucleic acid comprises the following formula:
5′ X₁X₂GGGX₃X₄ 3′
wherein X₁, X₂, X₃, and X₄ are nucleotides.

4. The method of claim 3, wherein at least one of X₃ and X₄ are a G.

5. The method of claim 3, wherein both of X₃ and X₄ are a G.

6. The method of claim 1, wherein the poly-G nucleic acid comprises the following formula:
5′ GGGNGGG 3′
wherein N represents between 0 and 20 nucleotides.

7. The method of claim 1, wherein the poly-G nucleic acid comprises the following formula:
5′ GGGNGGGNGGG 3′ (SEQ ID NO: 134)
wherein N represents between 0 and 20 nucleotides.

8. The method of claim 1, wherein the poly-G nucleic acid is administered mucosally.

9. The method of claim 8, wherein the poly-G nucleic acid is free an unmethylated CpG motif.

10. The method of claim 9, wherein the poly-G nucleic acid is selected from the group consisting of SEQ ID NOs: 95-133.

11. The method of claim 1, wherein the poly-G nucleic acid is administered systemically.

12. The method of claim 11, wherein the poly-G nucleic acid includes at least one unmethylated CG dinucleotide.

13. The method of claim 12, wherein the poly-G nucleic acid is selected from the group consisting of SEQ ID NO 46, 47, 58, and 61.

14. The method of claim 1, wherein the anti-microbial agent is selected from the group consisting of an anti-bacterial agent, an anti-viral agent, an anti-fungal agent, and an anti-parasitic agent.

15. The method of claim 14, wherein the anti-viral agent is selected from the group consisting of immunoglobulin, amantadine, interferon, nucleoside analogues, and protease inhibitors.

16. The method of claim 14, wherein the antiviral agent is selected from the group consisting of Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

17. The method of claim 14, wherein the anti-bacterial agent is an antibiotic.

18. The method of claim 14, wherein the anti-bacterial agent is a broad spectrum antibiotic.

19. The method of claim 14, wherein the anti-bacterial agent is a narrow spectrum antibiotic.

20. The method of claim 14, wherein the anti-bacterial agent is a limited spectrum antibiotic.

21. The method of claim 14, wherein the anti-bacterial agent is selected from the group consisting of cell wall synthesis inhibitors, cell membrane inhibitors, protein synthesis inhibitors, nucleic acid synthesis or functional inhibitors, and competitive inhibitors.

22. The method of claim 14, wherein the anti-bacterial agent is selected from the group consisting of natural penicillins, semi-synthetic penicillins, clavulanic acid, cephalolsporins, bacitracin, ampicillin, carbenicillin, oxacillin, azlocillin, mezlocillin, piperacillin, methicillin, dicloxacillin, nafcillin, cephalothin, cephapirin, cephalexin, cefamandole, cefaclor, cefazolin, cefuroxine, cefoxitin, cefotaxime, cefsulodin, cefetamet, cefixime, ceftriaxone, cefoperazone, ceftazidine, moxalactam, carbapenems, imipenems, monobactems, euztreonam, vancomycin, polymyxin, amphotericin B, nystatin, imidazoles, clotrimazole, miconazole, ketoconazole, itraconazole, fluconazole, rifampins, ethambutol, tetracyclines, chloramphenicol, macrolides, aminoglycosides, streptomycin, kanamycin, tobramycin, amikacin, gentamicin, tetracycline, minocycline, doxycycline, chlortetracycline, erythromycin, roxithromycin, clarithromycin, oleandomycin, azithromycin, chloramphenicol, quinolones, co-trimoxazole, norfloxacin, ciprofloxacin, enoxacin, nalidixic acid, temafloxacin, sulfonamides, gantrisin, and trimethoprim.

23. The method of claim 14, wherein the anti-bacterial agent is selected from the group consisting of Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Aziocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; and Zorbamycin.

24. The method of claim 14, wherein the anti-fungal agent is selected from the group consisting of imidazoles, FK 463, amphotericin B, BAY 38-9502, MK 991, pradimicin, UK 292, butenafine, chitinase and 501 cream.

25. The method of claim 14, wherein the anti-fungal agent is selected from the group consisting of wherein the anti-fungal agent is selected from the group consisting of Acrisorcin; Ambruticin; Amorolfine, Amphotericin B; Azaconazole; Azaserine; Basifungin; Bifonazole; Biphenamine Hydrochloride; Bispyrithione Magsulfex; Butoconazole Nitrate; Calcium Undecylenate; Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofungin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifungin Hydrochloride; Parconazole Hydrochloride; Partricin; Potassium Iodide; Proclonol; Pyrithione Zinc; Pyrrolnitrin; Rutamycin; Sanguinarium Chloride; Saperconazole; Scopafungin; Selenium Sulfide; Sinefungin; Sulconazole Nitrate; Terbinafine; Terconazole; Thiram; Ticlatone; Tioconazole; Tolciclate; Tolindate; Tolnaftate; Triacetin; Triafungin; Undecylenic Acid; Viridofulvin; Zinc Undecylenate; and Zinoconazole Hydrochloride.

26. The method of claim 1, further comprising administering to the subject an antigen.

27. The method of claim 26, wherein the antigen is a microbial antigen.

28. The method of claim 27, wherein microbial antigen is selected from the group consisting of a bacterial antigen, a viral antigen, a fungal antigen, and a parasitic antigen.

29. The method of claim 1, wherein the antigen is not conjugated to the poly-G nucleic acid.

30. The method of claim 1, wherein the anti-microbial agent is not a cytokine.

31. The method of claim 1, wherein the poly-G nucleic acid has a phosphorothioate modified backbone, and the poly-G nucleic acid is administered systemically.

32. The method of claim 1, wherein the poly-G nucleic acid is free of T-rich motifs and methylated CpG motifs.

33. A method for treating or preventing an infectious disease in a subject having or at risk of developing the infectious disease, comprising
administering to a subject in need of such treatment a CpG nucleic acid and an anti-microbial agent in an effective amount for treating or preventing the infectious disease, wherein the CpG nucleic acid is administered systemically.

34. The method of claim 33, wherein the effective amount is a synergistic amount.

35. The method of claim 33, wherein the anti-microbial agent is administered locally.

36. The method of claim 33, wherein the anti-microbial agent is selected from the group consisting of an anti-bacterial agent, an anti-viral agent, and an anti-fungal agent.

37. The method of claim 33, wherein the CpG nucleic acid is free of T-rich motifs, and methylated CpG motifs.

38. The method of claim 33, further comprising administering to the subject an antigen.

39. The method of claim 38, wherein the antigen is a microbial antigen.

40. The method of claim 39, wherein microbial antigen is selected from the group consisting of a bacterial antigen, a viral antigen, and a fungal antigen.

41. The method of claim 38, wherein the antigen is not conjugated to the CpG nucleic acid.

42. The method of claim 38, wherein the antigen is administered locally.

43. The method of claim 38, wherein the anti-microbial agent is not a cytokine.

44. The method of claim 38, wherein the CpG nucleic acid has a phosphorothioate modified backbone.

45. The method of claim 38, further comprising administering an adjuvant to the subject, provided the anti-microbial agent is selected from the group consisting of an anti-bacterial agent, and an anti-fungal agent.

46. A method for treating or preventing warts in a subject having or at risk of developing warts, comprising,
administering to a subject in need of such treatment, an immunostimulatory nucleic acid in an effective amount for treating or preventing the wart,
wherein the immunostimulatory nucleic acid does not have a phosphorothioate modified backbone.

47. The method of claim 46, wherein the immunostimulatory nucleic acid is a CpG nucleic acid.

48. The method of claim 46, wherein the immunostimulatory nucleic acid is a poly-G nucleic acid.

49. The method of claim 46, wherein the immunostimulatory nucleic acid is a T-rich nucleic acid.

50. The method of claim 46, wherein the immunostimulatory nucleic acid is a non-CpG nucleic acid.

51. The method of claim 46, further comprising administering to the subject an anti-microbial agent.

52. The method of claim 51, wherein the immunostimulatory nucleic acid and the anti-microbial agent are administered in an effective amount to synergistically treat or prevent the wart.

53. The method of claim 51, wherein the anti-microbial agent is an antiviral agent.

54. A method for prophylactically treating a subject at risk of developing the infectious disease, comprising
administering to a subject in need of such treatment an immunostimulatory nucleic acid having a phosphorothioate modified backbone, and an anti-microbial agent in an amount effective to inhibit the infectious disease,
wherein the immunostimulatory nucleic acid is free of a T-rich motif, a methylated CpG motif, and an unmethylated CpG motif.

55. The method of claim 54, wherein the effective amount is a synergistic amount.

56. The method of claim 54, wherein the anti-microbial agent is selected from the group consisting of an anti-bacterial agent, an anti-viral agent, an anti-fungal agent, and an anti-parasitic agent.

57. The method of claim 54, wherein the immunostimulatory nucleic acid is administered systemically.

58. The method of claim 54, further comprising administering an antigen to the subject.

59. The method of claim 58, wherein the antigen is a microbial antigen.

60. The method of claim 59, wherein microbial antigen is selected from the group consisting of a bacterial antigen, a viral antigen, a fungal antigen, and a parasitic antigen.

61. The method of claim 58, wherein the antigen is not conjugated to the immunostimulatory nucleic acid.

62. A method for preventing antibiotic resistance, comprising:
administering to a subject prior to, at the same time as or after the subject has received antibiotic therapy an effective amount of an immunostimulatory nucleic acid for preventing antibiotic resistance.

63. The method of claim 62, wherein the immunostimulatory nucleic acid is a CpG nucleic acid.

64. The method of claim 62, wherein the immunostimulatory nucleic acid is a T-rich nucleic acid.

65. The method of claim 62, wherein the immunostimulatory nucleic acid is a poly-G nucleic acid.

66. The method of claim 62, wherein the immunostimulatory nucleic acid is a nucleic acid having a phosphorothioate backbone modification.

67. The method of claim 62, wherein the immunostimulatory nucleic acid is administered before the antibiotic.

68. The method of claim 62, wherein the immunostimulatory nucleic acid is administered at the same time as the antibiotic.

69. The method of claim 62, wherein the immunostimulatory nucleic acid is administered after the antibiotic.

70. A method for preventing an allergic reaction in a subject receiving an anti-microbial agent, comprising
administering to a subject receiving an anti-microbial agent an immunostimulatory nucleic acid in an effective amount to prevent an allergic reaction to the anti-microbial agent.

71. The method of claim 70, wherein the anti-microbial is selected from the group consisting of an anti-bacterial agent, an anti-viral agent, an anti-fungal agent, and an anti-parasitic agent.

72. The method of claim 70, wherein the anti-microbial agent is an anti-bacterial agent.

73. The method of claim 70, wherein the anti-microbial agent is penicillin.

74. The method of claim 70, wherein the immunostimulatory nucleic acid is a CpG nucleic acid.

75. The method of claim 70, wherein the immunostimulatory nucleic acid is a T-rich nucleic acid.

76. The method of claim 70, wherein the immunostimulatory nucleic acid is a poly-G nucleic acid.

77. The method of claim 70, wherein the immunostimulatory nucleic acid has a phosphorothioate modified backbone.

78. The method of claim 74, wherein the immunostimulatory nucleic acid is administered systemically.

79. A kit comprising
at least one container housing an immunostimulatory nucleic acid, and
at least one container housing an anti-microbial agent, and
instructions for systemic administration of the immunostimulatory nucleic acid,
wherein the immunostimulatory nucleic acid is selected from the group consisting of a CpG nucleic acid, a poly-nucleic acid and a nucleic acid having a phosphorothioate modified backbone.

80. The kit of claim 79, wherein the at least one container housing an immunostimulatory nucleic acid is a sustained release vehicle.

81. The kit of claim 79, further comprising instructions for administering the immunostimulatory nucleic acid and the anti-microbial agent in an effective amount for inducing a synergistic immune response in the subject.

82. A composition, comprising:
an immunostimulatory nucleic acid and an antibiotic, formulated in a pharmaceutically-acceptable carrier and in an effective amount for preventing the development of antibiotic resistant strains of bacteria.

83. The composition of claim 82, wherein the antibiotic is selected from the group consisting of broad spectrum antibiotics, narrow spectrum antibiotics, and limited spectrum antibiotics.
